(19) United States
(12) Patent Application Publication
(10) Pub. No.: US 2005/0130124 A1
(43) Pub. Date: Jun. 16, 2005

(54) Phagemid display system

(76) Inventors: Erik Johan Wiersma, Ontario (CA); Donald Ian Hall Stewart, Ontario (CA)

Correspondence Address: Michael R Williams;Ade & Company, 1700-360 Main Street, Winnipeg, R3C 3Z3, CA

(21) Appl. No.: 10/491,550

(22) PCT Filed: Oct. 4, 2002
(86) PCT No.: PCT/CA02/01496

(30) Foreign Application Priority Data
Oct. 5, 2001 (US) 60326984
Nov. 26, 2001 (US) 60332531

(51) Int. Cl. C12Q 1/70; C12N 15/861

(52) U.S. Cl. 435/5; 435/457

(57) ABSTRACT

The present invention provides a novel helper phage and phagemid and phagemid display system that comprises an amber mutation in gene 3 of the helper phage so that it is not expressed in the non-permissive bacteria and an in-frame stop codon in the phagemid prior to the gene 3 coding sequence that prevents expression of g3p unless a foreign gene is inserted therein, thus preventing propagation of insert-less phagemids. This results in improved display of foreign gene products on individual virions, avoidance of virions lacking foreign gene inserts and the creation of large phage display libraries.

This application claims priority under 35 USC § 119(e) to Provisional Patent Application Ser. No. 60/326,984 filed on Oct. 5, 2001 and Provisional Patent Application Ser. No. 60/332,531, filed Nov. 26, 2001.

### FIELD OF THE INVENTION

The invention relates to helper phage/phagemid display system, to the components thereof and to the methods and uses thereof.

### BACKGROUND OF THE INVENTION

Phage display technology (PDT) is a highly versatile technique for studying interactions between biochemical molecules and for isolating polypeptides having a variety of binding or enzymatic activities [1-3].

PDT is a methodology established in the literature, which is used to express (or display) proteins on the outer surface of the capsid of bacteriophages. The principle is as follows: filamentous bacteriophages, or Ff phages, can be modified by genetic manipulation to package foreign genes into their capsids allowing the expression of the corresponding proteins as fusion proteins on the outside of the capsid. From a large collection of phages containing different foreign genes (a “library”) one can use affinity purification (or “biopanning”) to recover desired phage clones that interact with the molecule being used in biopanning. As an example, the foreign gene could encode an Fab fragment of an antibody, and when genetically fused to viral gene 3, the corresponding fusion protein, Fab-g3p (Fab-gene 3 protein), will be incorporated and displayed on viral capsids. An antigen is then used to biopan for phage clones expressing a Fab-g3p fusion protein with specific binding activity.

There are a variety of different types of PDT libraries. Originally phage systems were used to develop the libraries [4] (Also, see Ladner WO 90/02809). These systems utilize a single vector consisting of a modified phage genome comprising a foreign gene. Although, such systems are simple, it is difficult to make large libraries: the relatively large size of the vector, and other factors result in this vector being transformed into bacteria with a relatively poor efficiency. As such, phage systems have largely been replaced by different phagemid systems, which enable the creation of larger libraries and in some aspects, improved functionality compared to the original phage systems (summarized in Table 1).

In the first generation phagemid systems (such as U.S. Pat. No. 6,040,136 to Garrard et al, Mar. 21, 2000, and U.S. Pat. No. 6,127,132 to Breitling et al, Oct. 3, 2000) two vectors are necessary:
(a) A phagemid vector, which encodes for a fusion protein: i.e.: a foreign gene product (e.g. a Fab fragment) fused to a viral coat protein, typically g3p (gene 3 protein) but sometimes gene 8 protein; and
(b) a helper phage, which provides the necessary components for viral assembly (genes 1 through 10).

Although first generation phagemid systems are superior to phage systems in many aspects, they do not give efficient display of foreign gene products on the viral capsids—an important feature of PDT. This is due to helper phage expressing g3p, which preferentially become incorporated on viral capsids at the expense of g3p fusion protein (e.g. Fab-g3p) encoded by the phagemid.

This problem has been addressed in the past with what are described as second-generation phagemid systems. These systems differ from the first-generation phagemid systems in that the helper phage does not synthesize g3p. Without helper phage-encoded g3p, the only source of g3p is the phagemid vector (which express the protein encoded by the foreign gene fused to gene 3 (e.g. Fab-g3p). In these systems display levels are high and generally comparable to phage systems. However, such systems [5,7, 33] generally require three vectors: In addition to the helper phage and phagemid vector mentioned above, an additional g3p-producing vector is required to supplement the g3p-less helper phage when it is produced by its host bacteria.

To work well, many aspects of PDT must be optimized. As reviewed in Table 1, none of the current PDT systems address all problems. More specifically, the problems that need to be addressed are:
(a) The ability to create large libraries. The possibility of isolating a foreign gene product with the desired function increases with the size of the library.
(b) The ability to avoid creating insert-less clones. Libraries of any kind are a burden to the bacterial host, and tend to deteriorate as the host undergoes proliferation. Insert-less clones are less of a burden than other clones and will preferentially expand and dominate the library. To prevent insert-less clones when creating a library, one may utilize vectors which have multiple restriction sites [28, 18]. Moreover, insert-less clones can be avoided by: Using a vector that produces a toxin in the absence of an insert but inactive (or no) toxin in the presence of an insert [32]; Other similar approaches reviewed in this reference.
(c) The ability to minimize propagation of insert-less clones. In addition to avoid creating bacteria harboring insert-less phagemids, one may also prevent such clones, if created, from propagating as phage. Some phage systems (12, 13), and one phagemid system [14], have a vector design, which ensures that insert-less clones can not be packaged into infectious virions. Thereby, the propagation of insert-less clones is avoided.
   Finally, library diversity is better maintained if the foreign gene products are non-induced during most of the library propagation rather than being constitutively expressed. In current phage systems foreign genes are typically constitutively expressed whereas in phagemid systems expression is controlled.
(d) Phages should display as much foreign gene product as possible. In biopanning, it is easier to isolate the desired clones if a lot of foreign protein is displayed on each phage particle. This is distinct from having a large library of different clones. As detailed above, first generation phagemid systems give poor display, but both phage systems and second-generation phagemid systems demonstrate good display.
(e) The PDT system needs to be simple.
   Simpler biological systems tend to require less effort and be less prone to malfunction. In general, phage systems (which have one vector) are simpler than first generation phagemid systems (two vectors), which, again, are simpler than second-generation phagemid systems (three vectors [5, 6, 7, 33].
   Therefore, there is a need for a better phage/phagemid system that addresses the above-noted problems.

### BRIEF DESCRIPTION OF THE DRAWINGS

The invention will now be described in relation to the drawings in which:

FIG. 1 illustrates the structure of wildtype and mutant helper phage; M13K07 and Phaberge, respectively. FIG. 1A shows an overview of the phage genome. Numbers indicate the positions of the restriction sites that were used to create Phaberge from M13K07, as well as the translational start of gene 3. FIG. 1B is a more detailed illustration of the sequence that was mutagenized in one embodiment of the invention.

FIG. 2 illustrates several points: “A” indicates how vectors were constructed in a chronological order, whereas in “B”, the order of presenting the vectors is based on their similarity. Phagemid pMAB2 (FIG. 2A) and its derivatives were constructed in this work. Vector pTIM1 and its predecessors have been described in the prior art. FIG. 2B is a schematic illustration of the phagemid vectors that were tested for function in this patent disclosure. Vector pMAB29 is illustrated in full. For vectors pMAB66, pMAB77, pMAB103 and pMAB87 only the parts that differ between vectors have been illustrated. . Bold, large font in FIG. 2B indicates differences between phagemids as follows: pMAB77 differs from pMAB29 in that it lacks a c-myc tag, has a rho-dependent terminator and that it has motifs for conversion to expression of soluble, poly-histidine tagged Fab fragments. (The g3p gene can be removed by NheI-digestion and self-ligation, bringing V_{H}C_{H}1 in frame with a stretch encoding for a hexa-histidine tag). pMAB66 differs from pMAB77 in the length of the g3p gene: residues 211-406 (of the leader-less g3p), or residues 3-406, respectively. pMAB103 differs from pMAB77 in that it uses a different plasmid origin of replication. pMAB87 differs from pMAB77 in that it lacks both VκCκ-insert and V_{H} insert, and that the g3p gene is preceded by a translational stop codon. The lowest section of FIG. 2B is a detailed view of the V_{H} cloning site of vector pMAB87. The translation stop codon is in bold. The V_{H} cloning site contains an extra RE (restriction enzyme) site, AscI, which is used to avoid creation of insert-less clones by reducing self-ligation of vector that has not been sufficiently digested with REs MunI and SalI.

FIG. 3 illustrates the production of phage virions and their display of Fab-g3p under different conditions. The figure illustrates bacterial cells harboring phagemid vector and helper phage genome. Gene 3 expression is indicated by a bold hooked arrow and absence of expression is indicated by a “X”.

FIG. 4 illustrates the results of a PFU (plaque-forming unit) assay, measuring the content of M13K07 or Phaberge in crude helper phage preparations. The Figure also illustrates how these helper phage replicate when indicator cells of different genotypes are used in the PFU assay.

FIG. 5 is the sequence of gene 3 of helper phage clone 4B. The Figure is a chromatogram obtained by DNA sequencing in the sense direction.

FIG. 6 is a Western blot analysis of virally associated g3p. Phagemid virions were analyzed as described in Section A.1.4.2. For preparation made by Phaberge and by M13K07, equal numbers of virions were loaded. The identities of the two bands were deduced by molecular mass markers, and by the fact that the upper band (“Fab-g3p”) also probed with anti-κ reagent (data not shown).

FIG. 7 illustrates an ELISA to determine antigen specificity of three unique phagemid clones: numbers 2, 13 and 14. Wells of an ELISA plate were coated with either tetanus toxoid (“TT”), bovine serum albumin (“BSA”) or human platelet protein GPIIbIIIa (“2b3a”). Binding of phagemid virions to each antigen was tested as described in Section A.1.4.1.

### SUMMARY OF THE INVENTION

The current invention is an improvement on the prior phagemid systems in obtaining better display of foreign protein on phage particles and in avoiding propagation of insert-less phages. A novel approach to ablating g3p expression from helper phage is utilized, thereby improving display of foreign gene products. In another aspect, the invention provides a helper phage comprising a conditional mutation at the 3′end of g3p wherein the g3p can be expressed in a conditional host, but is not expressed in a non-conditional host.

In one embodiment, the conditional mutation causes minimal or no polar effects to downstream genes. In another embodiment, the helper phage is a M13K07 helper phage. In one aspect, the mutation is an amber mutation, preferably at the late the 3′end of gene 3, most preferred at Q350. The g3p of the helper phage can be expressed in a permissive host. In one embodiment, the permissive host is Sup E E. coli. In addition the phagemid vector used in this invention has combined several features, which together improve functionality beyond what has been achieved with previously reported PDT systems. In one embodiment, the invention provides a phagemid comprising a gene 3, a restriction site to enable the insertion of a foreign gene in-frame with the gene 3 to create a g3p fusion protein when expressed, and a sequence feature that prevents g3p synthesis in the absence of an inserted foreign gene. In another embodiment, sequence feature of the phagemid is an in-frame stop codon prior to the g3p gene. In yet another embodiment the phagemid is pMAB87, preferably comprising the SEQ. ID NO. 7 with the replacement of bases 237-1648 with SEQ. ID. NO. 17 as described in section A.1.5.9, herein below. When utilized together with the above mentioned helper phage, no g3p will be synthesized by either vector if a foreign gene insert is absent: Because of the lack of g3p, insert-less phagemid clones will not produce infectious phage, and such deficient clones (but not insert-containing clones) will thus be unable to propagate.

In another aspect of the invention, the invention provides a phagemid display system comprising a phagemid as described above wherein a foreign gene is inserted into the phagemid, and a helper phage as described above, to enable the protein expressed by the foreign gene to be displayed on the bacteriophage. In yet another aspect, the invention provides a peptide library that can be screened with molecules or peptides having potential binding activity to the foreign gene product displayed on phage virions. In one embodiment the protein is an antibody and the molecule or peptide is a potential antigen or vice versa.

According to a first aspect of the invention, there is provided a helper phage for phage display comprising a conditional mutation in a filamentous phage viral coat protein gene wherein the conditional mutation causes minimal or no polar effects to downstream genes.

According to a second aspect of the invention, there is provided a phagemid vector comprising: gene 3 from filamentous bacteriophage; and a cloning site for inserting a nucleic acid molecule therein in-frame with gene 3 for creating a g3p fusion protein when expressed.

According to a third aspect of the invention, there is provided a phage display system comprising:

a helper phage for phage display comprising a conditional mutation in a filamentous phage gene 3 wherein the conditional mutation causes minimal or no polar effects to downstream genes; and

a phagemid vector comprising:
gene 3 from filamentous bacteriophage;
a cloning site for inserting a nucleic acid molecule therein in-frame with gene 3 for creating a g3p fusion protein when expressed; and
a sequence feature that prevents g3p synthesis in the absence of an inserted nucleic acid molecule.

According to a fourth aspect of the invention, there is provided a method of creating a phagemid display system, comprising:

providing a helper phage for phage display comprising a conditional mutation in a filamentous phage gene 3 wherein the conditional mutation causes minimal or no polar effects to downstream genes.

providing a phagemid vector comprising:
gene 3 from filamentous bacteriophage; and
a cloning site for inserting a nucleic acid molecule therein in-frame with gene 3 for creating a g3p fusion protein when expressed; and

infecting a bacterial host with the phagemid and the helper phage.

According to a fifth aspect of the invention, there is provided a method of screening for compounds binding to a target molecule using a phagemid display system, comprising:

providing a helper phage comprising a conditional mutation in a filamentous phage gene 3 wherein the conditional mutation causes minimal or no polar effects to downstream genes;

providing a phagemid vector comprising:
gene 3 from filamentous bacteriophage;
a cloning site for inserting a nucleic acid molecule therein in-frame with gene 3 for creating a g3p fusion protein when expressed; and
at least one nucleic acid molecule encoding a peptide inserted into the cloning site in frame with gene 3; and

infecting a bacterial host capable of suppressing the conditional mutation with the phagemid and the helper phage;

recovering the phagemid and the helper phage;

infecting a non-suppressing bacterial host with the phagemid and the helper phage;

growing the non-suppressing bacterial host under conditions wherein the phagemid is expressed, thereby producing a phage display library;

incubating the target molecule and the phage display library under conditions which promote peptide binding; and

detecting peptide binding.

According to a sixth aspect of the invention, there is provided a nucleic acid molecule encoding a peptide capable of binding to a target molecule identified according to the method of claim 16.

According to a seventh aspect of the invention, there is provided a peptide capable of binding to a target molecule identified according to the method of claim 16.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

Described herein is a novel phage system for use in phage display. As described below, the system comprises a helper phage and a phagemid.

Specifically, the helper phage includes a conditional or suppressable mutation, for example, a nonsense mutation, for example, an amber or ochre mutation, within a filamentous bacteriophage viral coat protein, for example, gene 3 or gene 8. As will be appreciated by one of skill in the art, as a result of this arrangement, the helper phage expresses gene 3 when grown in a suitable host bacterium which suppresses the nonsense mutation, for example, Sup E E. coli, but is not expressed in a non-conditional host. In some embodiments, the mutation is one that results in minimal polar effects, that is, minimal effects of the translation of downstream genes. In some embodiments, the mutation is in the latter half, or latter third or is proximal to the 3′ end of gene 3.

The phagemid comprises a cloning site upstream of a viral coat protein, for example gene 3 or gene 8 so that nucleic acids encoding (poly)peptides of interest can be inserted therein in frame with the viral coat protein so that a foreign protein-viral coat protein product is produced. As will be appreciated by one of skill in the art, any suitable nucleic acid may be inserted into the phagemid, for example, although by no means limited to nucleic acid encoding peptide, peptide fragments, or cDNA or peptide libraries. In some embodiments, the cloning site is arranged such that expression of gene 3 is prevented unless a foreign nucleic acid molecule is inserted into the cloning site. In some embodiments, this expression inhibition signal comprises an in-frame stop codon preceding gene 3, although other means of preventing expression known in the art, for example, structural elements, may also be used.

In use, a library is constructed as described below using the above-described phagemid. The phagemid and the helper phage described above are propagated in a conditional host as described herein which suppresses the conditional mutation in gene 3 of the helper phage. As a result of this arrangement, the helper phage provides the necessary components for viral assembly. Phagemid and helper phage are then recovered and grown in a non-suppressing host. As a consequence, the mutation in helper phage gene 3 is not suppressed, meaning that gene 3 is not expressed and there is no viral assembly, meaning that no further helper phage is produced. Similarly, gene 3 is not produced in phagemid lacking an insert in the cloning site, as discussed herein. Thus, only phagemid containing a nucleic acid encoding a foreign peptide of interest propagate, as discussed below.

As discussed below, the system is used in phage display. Accordingly, peptide or cDNA libraries may be inserted into the cloning site of the phagemid and the phagemid may be produced as described herein. The phagemid can thus be used to produce a library which can be screened for interaction with a target molecule or molecule of interest. That is, the phagemid library is expressed in a suitable host, the molecule of interest is incubated with the library and binding between the molecule of interest and foreign gene—gene 3 fusions is detected using means known in the art. According to another aspect of the invention, a method of screening and targets identified by this method are provided, as discussed below.

The present invention provides a novel phagemid system for use in phage display. The problems which have been addressed in innovative ways fall into two areas:
(a) Obtaining better display of foreign protein on phage particles. The solution to this is in a novel helper phage; and
(b) Avoiding propagation of insert-less phages. The solution to this is both in a novel helper phage and a novel phagemid vector.
   Most phage display systems utilize phagemid vectors where the protein of interest (POI) is genetically fused to gene 3 protein (“POI-g3p”). Although such systems work relatively well, they have deficiencies: Such systems utilize a helper phage whose synthesis of g3p has negative consequences:
Phage particles do not display a high level of POI-g3p. When virions are assembled, POI-g3p is poorly incorporated—it is displaced by helper phage-encoded g3p.
Both insert-less and insert-containing phagemids (i.e. both useless and useful phagemids) are assembled into functional virions. Since the helper phage-encoded g3p is always present, infectious virions will be formed regardless of whether or not the phagemid encodes for a useful form of g3p.

Previous studies show that the first problem can be solved by using a mutant helper phage whose entire gene 3 has been deleted. Although useful, such helper phage are usually produced at a low level and may also suffer from leaky g3p-production, genetic instability and polar effects.

These problems are addressed by the present invention by introducing a discrete and conditional mutation into gene 3 of helper phage M13KO7: Q350Amber. The mutant helper phage, “Phaberge”, was found to have similar functionality as the wildtype helper phage M13K07 when produced in a permissive E. coli host: SupE+. However, when such helper phage were used to infect a non-permissive E. coli carrying a phagemid vector, Phaberge was found to have better functionality than M13K07:

POI-g3p was displayed at a significantly higher level when using Phaberge instead of M13K07.

Phaberge had a very strong discrimination in that insert-less phagemids were packaged into functional virions with extremely poor efficiency, whereas insert-containing phagemid virions were produced at similar, high levels as with M13K07. Thus, it was found that the novel helper phage had improved functionality, generally useful in phagemid vector systems.

I. Novel Helper Phage:

Phagemid systems can display more foreign protein if the helper phage does not express g3p. However if the helper phage do not contain functional g3p on their capsids they are unable to infect bacteria. Simply inactivating the helper phage's gene 3 is thus not appropriate. The helper phage particles must be assembled in the presence of g3p to be infectious, but once they have infected the phagemid host it is preferable if the helper phage do not express g3p.

Thus in one embodiment, the invention provides a helper phage that has a conditional or suppressible mutation in gene 3. In one embodiment the mutation is located in a position that results in minimal polar effects on downstream genes. In a preferred embodiment, the mutation is in the 3′ end of gene 3, most preferably in the late 3′ end of the gene 3.

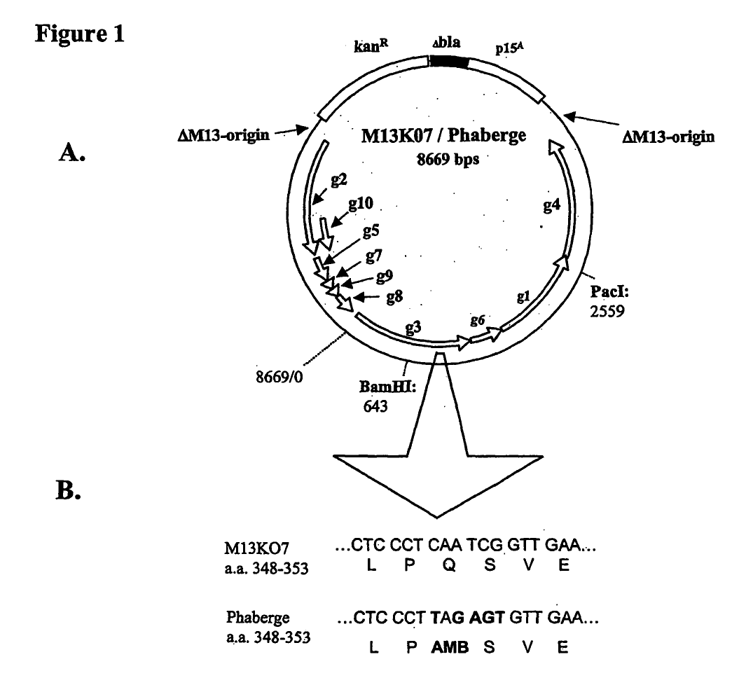

In a preferred embodiment, in order to turn on and off helper phage g3p synthesis, a conditional, or suppressible mutation was introduced in gene 3 of helper phage M13KO7. In one embodiment, the mutation was in the most C-terminal glutamine codon of gene 3 which was exchanged for an amber stop codon (FIG. 1). This mutant helper phage, named Phaberge, is produced in a permissive host, such as having genotype SupE (E. coli strain XL-1Blue MRF′). The SupE genotype allows for expression of full-length g3p, and hence assembly of functional infectious phage. However, after their production, Phaberge is used to infect a phagemid host of non-permissive genotype (i.e. does not have SupE, e.g.: E. coli strain TOP10F′). Thus, in this new host, only the phagemid (not the helper phage) will make full length, functional g3p, and this gives good display of Fab-g3p (Table III).

II. A Novel Phagemid Vector: pMAB87

The phagemid vector of the invention has a functional gene 3 and at least one restriction site that enables insertion of a gene encoding a (poly-)peptide of interest (“POI”) in frame with the gene 3 to result, upon expression of the gene, in a fusion protein—“POI-g3p”. In a preferred embodiment, the phagemid vector has at least two, and preferably two, dissimilar restriction sites that enables insertion of a gene encoding a desired (poly-)peptide in frame with the gene 3 to result, upon expression of the gene, in a fusion protein—“foreign-peptide-g3p”. The phagemid is so constructed to prevent expression of g3p unless a foreign gene is inserted therein. In one embodiment, this is achieved by an in-frame stop codon preceding gene 3.

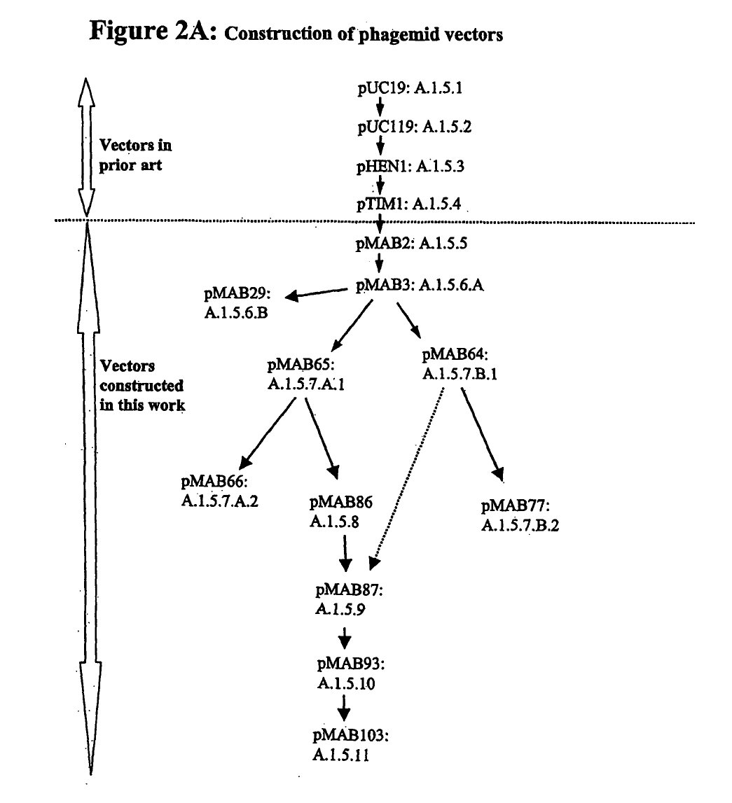

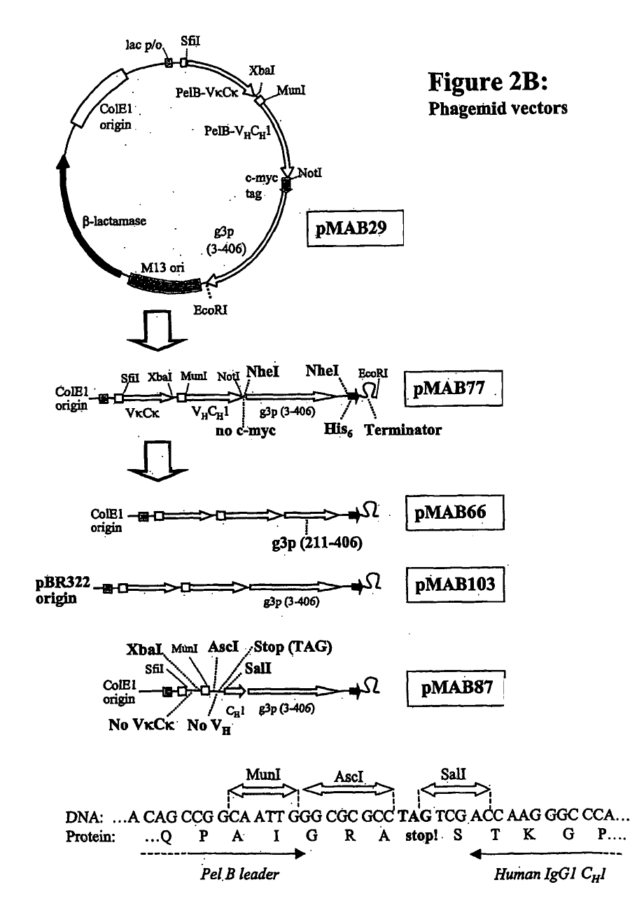

In one embodiment, phagemid vector pMAB87 (FIG. 2) is used for expression of antibody Fab fragments, although a person skilled in the art would appreciate that the vector could be used to express any other peptides. For Fab genes, a Fab-g3p fusion protein is expressed after insertion of V_{L}C_{L} and V_{H} genes in their respective cloning sites of the vector.

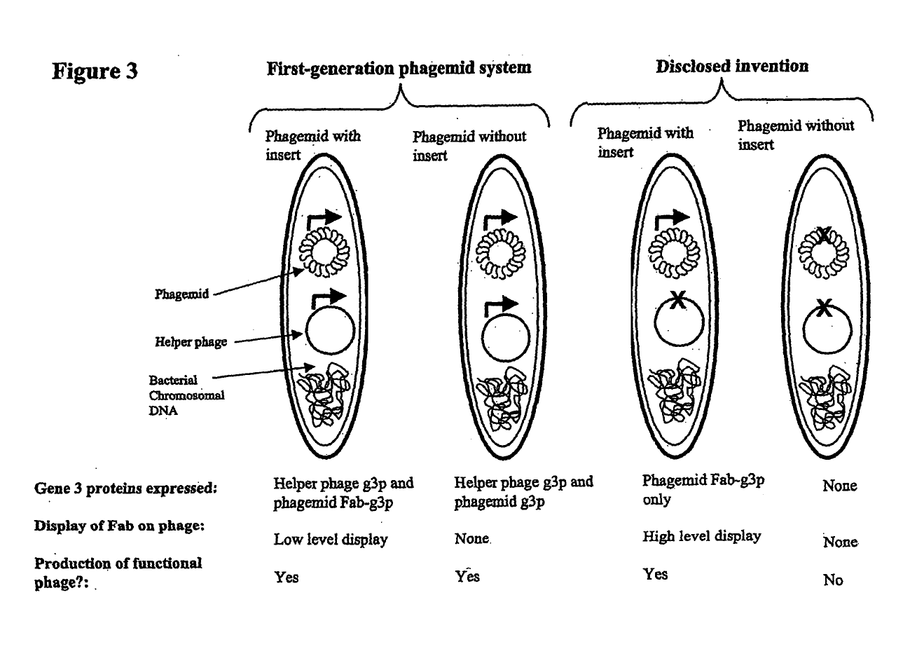

pMAB87's cloning site for V_{H} has a feature, which ensures that only phagemid clones containing a V_{H} insert give viable phage. This site (FIG. 2B, bottom) contains an in-frame translational stop codon, which precedes gene 3 and prevents expression of g3p unless the stop codon is replaced by a foreign gene, such as V_{H}. Since only phagemid, not helper phage, can express g3p in this system, the only way any g3p can be expressed is if V_{H} (or another foreign gene) is inserted in the cloning site (FIG. 2B, bottom). Since g3p is required for assembly of infectious phage, viable phages will only be produced if the phagemid contains a V_{H} insert (or other foreign gene insert). The insert-less clones are unable to produce infective phage (FIG. 3) and will not be able propagate since they are non-infectious.

III. Problems and Solutions, Overview

(a) Obtaining a Helper Phage with Better Functionality

Both phage systems and second-generation phagemid systems exhibit good display of foreign protein on phage particles (Table 1). Phage systems utilize a vector type that is different from both second-generation phagemid and also from that described in this invention. In addition the phage system approach has additional distinct disadvantages as summarized in Table 1.

In phagemid systems, the key to obtaining better display lies in the ability to regulate the helper phage's g3p synthesis, i.e.: to produce helper phage virions having g3p on their capsid, yet avoid having the same helper phage synthesizing g3p after they have infected a phagemid-bearing host. Three other research groups have presented separate solutions to this problem. In all three cases, the solution was to delete essentially the entire gene 3 from the helper phage genome and having the helper phage host synthesize gene 3: In the first two systems (one described by Griffiths et al. and McCafferty et al [5, 6]; the other one by Larocca et al. and Rakonjac et al. [33, 34]) the host that harbors the gene 3-deficient helper phage also contains a plasmid encoding g3p. In the third system, described by Rondot et al. [7] the helper-phage host has integrated gene 3 in its chromosomal DNA.

The present invention differs from all these approaches as in the present case the helper phage has a conditional mutation at the 3′ end of gene 3, rather than a complete deletion of gene 3. Also it differs from others in that it does not need the helper phage host to synthesize g3p. In this invention the host provides permissive conditions (i.e. SupE) allowing expression of full-length g3p from the mutated helper phage. The advantages of this are: First, since the helper phage does produce full-length g3p in its host, the host does not need to carry an extraneous vector encoding g3p. Thus, a simpler system is obtained. Second, helper phage gene 3 is under its natural genetic control elements. This should avoid over- or under-expression of g3p, both of which can have negative effects on the host and its production of helper phage.

Bass and co-workers [8] constructed a mutated variant of helper phage M13KO7: The amino acid mutation E196→amber (stop codon) was introduced in gene 3. (The article incorrectly states that the mutation is E197→amber). The present invention differs from that of Bass et al. in two aspects: First, the publication of Bass et al. did not mention or show any novel utility of the mutated helper phage beyond what was found for the un-mutated helper phage;

Second, their mutated helper phage was clearly inferior to M13KO7 in supporting production of phagemid virion particles. This may be due to the fact that the E→amber mutation is located far from the 3′ end of gene 3, and likely has polar effects on downstream helper phage genes [9-11]. The present invention differs from this prior art in that the mutation (Q350→amber) is in the late, 3′ end of gene 3 and gives in our hands no, or very minimal polar effects.

There are also previous publications describing mutant filamentous phage [9, 11, 37, 38] containing amber stop codons in gene 3. However, these do not constitute relevant prior art since: first, these constructs were made before PDT was invented and have not been considered for PDT. The stated intention was instead to study filamentous phage and their genes as a biological model system. Second, these modified phage are not suitable for PDT since unlike helper phage, they have a wildtype origin of replication. A defect origin, which is present in helper phage, is necessary both to reduce the stress that viral replication causes to the host bacterium, and also for helper phage to package phagemid ssDNA into virions at expense of its own ssDNA.

(b) Avoiding Propagation of Insert-Less Phages

In the present invention, insert-less phagemid clones do not produce significant amounts of infectious phage particles, since g3p synthesis is disallowed in such insert-less clones. Two critical features give the system of the invention this trait:
(1) There is only one source of functional g3p—the vector utilized for expression of a foreign gene/gene 3 fusion protein.
(2) The cloning site for the foreign gene contains a sequence feature (stop codon) that prevents g3p synthesis in the absence of an inserted foreign gene.

Some phage systems [12, 13] have both these features and have been used to prevent propagation of phage particles that lack an inserted foreign gene. The disclosed invention differs from these by being a phagemid system which in many other aspects have better functionality than phage vector systems (Table I).

The disclosed invention also differ from second-generation phagemid systems [5, 7, 33] which have the first, but not the second of the two features described above. Such second-generation phagemid systems do not prevent viral propagation of insert-less clones. Also, it is not obvious that such systems can be re-designed to prevent viral propagation of insert-less clones: Some, if not all, of these systems suffer from leakiness in g3p production, and it is therefore not evident that infectivity (and thereby selection) can be controlled by regulating phagemid-encoded g3p.

A phagemid system by Kristensen and Winter [14] prevents viral propagation of insert-less clones, despite having only the second of the two features. Although propagation of insert-less clones is avoided, this system has quite limited utility; only short, protease-resistant foreign gene products can be displayed. The publication describes a phagemid, pDK2, in which the multiple cloning site (MCS) for inserting POI genes is located in the middle of g3p. Only short peptides can be displayed in this case since longer ones will intervene with g3p and disrupt its ability to mediate infection. This is different from the phagemid in the present invention, pMAB87, where the location of MCS is 5′ (N-terminally) of g3p, and which allows for insertion of both large and small polypeptides without interfering with g3p function. In addition the helper phage described by Kristensen and Winter encodes for g3p, and therefore the display level is expected to be fairly low.

Yazynin et al. [32] described a phagemid vector where construction of insert-less clones is avoided. Our system is conceptually different from this: Features number “1” and “2” above prevent viral propagation of insert-less clones, whereas the system by Yazynin et al. prevent the initial creation of bacterial transformants carrying insert-less clones.

The prior art contains examples where synthesis of a vector-encoded protein is critically dependent on having a POI gene inserted into the vector, e.g. plasmid systems for α-complementation of the enzyme β-galactosidase [20]. In these cases, insert-containing bacterial colonies can be enzymatically identified in situ and manually selected. Although this feature is reminiscent of feature “2” above, it differs from the disclosed invention as follows: selection by α-complementation is not useful for PDT libraries as such libraries typically have 10⁷-10¹⁰ clones, and it is therefore not practical (even with robots) to pick such a large number of clones. In the disclosed invention, selection does not require identification or picking of bacterial colonies. Instead it is based on that insert-less phagemids cannot be packaged into functional virions—selection is built into the host-vector system itself.

(c) Obtaining Better Display of Foreign Protein on Phage Particles

Compared to other PDT systems having good display, the present invention has the following advantages:
(1) The invention is a phagemid system. Compared to phage systems this enables creation of larger libraries and makes possible the use of regulated expression of foreign genes, which is important for maintaining library diversity.
(2) As compared to second generation phagemid systems our invention uses a mutated helper phage which has better functionality:
(i) It is a simpler system in that helper phage host does not need to encode gene 3.
(ii) The mutant helper phage virions (Phaberge) are produced with similar high efficiency as corresponding wildtype helper phage (at least 10¹⁰ PFU/mL). The mutant helper phages used in existing second-generation phagemid systems [5-7] are typically produced at several ¹⁰log-units level lower.
(iii) Preparations of Phaberge helper phage does not appear to give significant leakiness of g3p synthesis in the phagemid host. Such leakiness might occur if the helper phage host has a plasmid encoding for g3p, which can be packaged and transferred to the phagemid host. This is expected to be the case in the system of Griffiths et al and McCafferty et al [5, 6] and in the system described by Larocca et al. and Rakonjac et al. [33, 34].
   (d) Avoiding Insert-Less Clones

The present system has features that both minimize the occurrence of insert-less clones when constructing a library and prevent any insert-less phages from propagating. Only one other phagemid system can prevent propagation of insert-less phage [14], but as above this system has very limited utility allowing display of only short, protease-resistant foreign gene products.

IV. Applications

Phage display technology involves the expression of a heterologous, (poly-)peptide library on the surface of bacteriophages. Applications of this technology include the isolation monoclonal antibodies specific for a predetermined antigen, identification of other types of interacting polypeptides, such as: mapping pairs or clusters of naturally occurring proteins that interact with each (i.e. proteomics) or de-novo-constructed artificial (poly)peptides with selective binding activity; polypeptides with enzymatic activity. This can be achieved by incubating the bacteriophage displaying relevant (poly)peptide with appropriate target molecule, as exemplified in Section A.1.6 and references [29, 30, 31]. The same procedures can also be used to select and isolate for the genes for the displayed peptide. Isolated (poly)peptide genes may have clinical utility, such as expression and usage of soluble monoclonal antibodies to treat or detect cancer, infectious diseases, hemostatis/thrombosis, autoimmune diseases or transplantation incompatibilities.

The following non-limiting examples are illustrative of the present invention:

### EXAMPLES

A. Material and Methods

1.1 Construction of Mutated Helper Phage

Overlap extension PCR [24] was used to insert the mutation Q350→amber into gene 3 of helper phage M13KO7 (Amersham-Pharmacia [23]; see FIG. 1). To generate a 1.9 kb mutated fragment, encompassing the BamHI-PacI region of M13KO7, we used the following four PCR primers (see [6] for naming of primers): A: CTG GCT TTA ATG AGG ATC CAT TCG TTT GT [SEQ. ID. No. 1]; B: ATT CAA CAC TCT AAG GGA GGG AAG GTA AA [SEQ. ID. No. 2]; C: CTC CCT TAG AGT GTT GAA TGT CGC CCT TTT GTC [SEQ. ID. No. 3]; D: TGC TTC TGT AAA TCG TCG CTA [SEQ. ID. No. 4]. The mutated PCR fragment was inserted into the TOPO-TA shuttle vector (Invitrogen). After verification of the DNA sequence, this PCR-derived BamHI-PacI fragment was digested out of the TOPO-TA backbone and inserted into the BamHI-PacI backbone fragment of M13KO7. After ligation and transformation into E. coli XL-1 Blue MRF′ (Stratagene), a plaque assay (Section A.1.2) was used to identify transformants able to produce replicating helper phage. Plaque-forming transformants were subjected to further characterization as detailed in “Results” section.

1.2 PFU and CFU Assays

PFU (plaque forming unit) and CFU (colony forming unit) assays were performed by standard microbiological techniques [19, 20]. Briefly, indicator bacteria were grown to mid-log phase (A₆₀₀ of 0.6-0.8) and infected with a dilution series of either replication-competent helper phage (PFU assay) or phagemid virion, conferring ampicillin-resistance (CFU assay).

In the PFU assay, infected bacteria were mixed with melted 2×YT soft agar, and spread on petri dishes containing 2×YT agar. After overnight incubation, the number of plaques was determined. PFU assays used either TOP10F′ (Invitrogen/GibcoBRL) or E. coli XL-1 Blue MRF′ (Stratagene) bacterial strains as indicator cells.

In the CFU assay, infected bacteria were spread directly on agar plates containing 2×YT+1% (w/v) glucose+100 □g ampicillin/mL. After overnight incubation, the number of colonies was determined.

1.3 Production and Purification of Phage

Helper phage and phagemid virion were prepared essentially according to standard methods [20] [25] [26].

To prepare helper phage, infected bacteria were grown overnight in 2×YT media. The bacterial culture was heat killed (65° C. for 10 minutes) and supernatant harvested by centrifugation (10 minutes, 4,000×G). This helper phage preparation was aliquoted without further purification, and stored at −20° C.

To prepare phagemid virion, phagemid-containing bacteria were grown at 37° C. in liquid media (2×YT+1% (w/v) glucose+1000 □g ampicillin/mL) and infected with an excess of helper phage (either R408, VCS-M13, M13KO7 or Phaberge; see below) at mid-log phase (A₆₀₀ of 0.6-0.8). After infection for 30 minutes at 37° C., bacteria were centrifuged and resuspended in 2×YT liquid media containing 1000 □g ampicillin /mL. Infected bacteria were grown overnight at either 37° C. or 30-32° C. (see below). Supernatant was then clarified by centrifugation, after which phagemid virion was purified by two consecutive precipitations with PEG-NaCl.

1.4 Immunoassays

1.4.1 Phage ELISAs

To assay viral display of tetanus toxoid (TT)-specific Fab-fragments, a standard 96-well ELISA plate was coated with 5 μg/mL of TT (Statens Serum Institut, Denmark), diluted in 1×PBS+0.03% NaN₃. Alternatively, plates were coated with either mouse-anti-fd/f1 (Research Diagnostics, USA) or mouse-anti-pIII (Mobitec, USA) (both at 5 μg/mL) to determine number of phage particles or, coated with 1% BSA to determine non-specific binding. Coating was done for 2 hours at 37° C. or overnight at 4° C. All incubation steps were followed by three washes in 1×PBS+0.05% Tween20. After coating, wells were blocked with 1×PBS+1% BSA+0.03% NaN₃. Purified phagemid virion was applied in a serial dilution, using 1×PBS+1% BSA+0.03% NaN₃ as diluent and incubated 2 hours at 37° C. with gentle shaking or overnight at 4° C. Two alternative detection systems were used, each using reagents diluted 1:1,000 in 1×PBS+1% BSA+0.03% NaN₃ and incubated at one hour and 37° C. at each step. One system used sheep-anti-fd antibody (Seramun Diagnostics, Germany), followed by alkaline phosphatase(AP)-conjugated rabbit anti-sheep IgG (Jackson Laboratories, USA). The other system used biotin-conjugated mouse anti-fd antibody f1 (Research Diagnostics, USA) followed by AP-conjugated streptavidin (Jackson). After washing plate, substrate solution [27] was added and absorbance at 405 (“A₄₀₅”) nm was determined.

In Tables II and III anti-TT display of various samples is compared with a reference sample: For Table II, the reference sample was phagemid virion produced under standard conditions (see footnote 1 of the Table), and for Table III, the reference was phagemid virion produced using M13K07. To compare the display of anti-TT Fab of test and reference samples, the following formulae were used:

First, the anti-TT ELI SA titer was normalized for content of phage:
A=(Anti-TT-titer)/(Phage titer)
The “Anti-TT-titer” is the reciprocal of the dilution of phagemid virion which gives either 50% (Table II) or 25% (Table III) of maximal A₄₀₅ in the anti-TT ELISA. The “Phage titer” is either the number of CFU/mL or: the reciprocal of the dilution of phagemid virion which gives or 25% (Table III) of maximal A₄₀₅ in the anti-phage sandwich ELISA. Finally, the relative level of display of the test sample is expressed as a percentage of that of the reference sample using the following formula:
Difference in display=100×(Aₜₑₛₜ/A_{ref})
“Aₜₑₛₜ” is “A” from the first formula, calculated for the test sample and “A_{ref}” is “A” from the first formula, calculated for the reference sample.
1.4.2 Western Blot

Standard methods were used for visualizing g3p and κ-containing Fab-g3p by Western blot [35]. Briefly, 40 μL of different preparations of phagemid virions were separated by SDS-PAGE under non-reducing on a 10% acrylamide gel. After blotting onto nitrocellulose filter paper, probing was done for either g3p, using a mouse anti-g3p antibody (pSKAN3, Mobitech) followed by horse radish peroxidase (HRP-)conjugated goat-anti-mouse-IgG (Jackson), or for human □□chain using goat-anti-human-κ followed by HRP-conjugated goat-anti-mouse-IgG (Jackson). In both cases, Pierce Supersignal HRP Substrate was used for chemiluminescence detection

1.5 Construction of Phagemid Vectors

Many phagemid vectors were constructed using standard molecular biology techniques [19, 20] as briefly described in Sections 1.5.1-1.5.11 and FIG. 2. These cloning steps where done in a sequential fashion, making one or several alterations at each consecutive cloning step. Vectors pMAB29, pMAB77, pMAB66 and pMAB103 contain inserted gene fragments encoding for a fully human Fab fragment specific for tetanus toxoid (TT). These gene fragments were isolated by RT-PCR cloning from the human hybridoma cell line 9F12 [21, 22], obtained from ATCC, VA, USA.

1.5.1:

Name of vector: pUC19
Literature reference: Norrander et al., Gene, vol. 26, p. 101, 1983
Sequence reference: http://www.ncbi.nlm.nih.gov/Genbank/, access number M77789 [SEQ. ID. No. 5]
   1.5.2:
Name of vector: pUC119
Alteration from pUC19: Insertion of phage M13 origin of replication (“IG-region) into pUC19
Literature reference: Vieira and Messing, Methods Enzymol., vol. 153, p. 3, 1987.
Sequence reference: http://www.ncbi.nlm.nih.gov/Genbank/, access number U07650 [SEQ. ID. NO. 6]
   1.5.3:
Name of vector: pHEN1
Alteration from pUC119: Insertion of g3 from phage vector fd-tet-DOG-1. Also, multiple changes at 5′ end of inserted g3: Exchanged g3 leader for pelB leader; Introduced RE sites between pelB leader and structural part of g3; Introduced c-myc peptide tag and amber stop codon immediately 5′ of structural part of g3

Literature reference: Hoogenboom et al., Nucleic Acid Res., vol. 19(15), p. 4133, 1991,

| | |
|---|---|
| AGCGCCCAATACGCAAACCGCCTCTCCCCGCGCGTTGGCCGATTCATTAATGCAGCTGGC | SEQ. ID. NO. 7 |
| ACGACAGGTTTCCCGACTGGAAAGCGGGCAGTGAGCGCAACGCAATTAATGTGAGTTAGC | |
| TCACTCATTAGGCACCCCAGGCTTTACACTTTATGCTTCCGGCTCGTATGTTGTGTGGAA | |
| TTGTGAGCGGATAACAATTTCACACAGGAAACAGCTATGACCATGATTACGCCAAGCTTG | |
| CATGCAAATTCTATTTCAAGGAGACAGTCATAATGAAATACCTATTGCCTACGCAGCCGC | |
| TGGATTGTTATTACTCGCGGCCCAGCCGGCCATGGCCCAGGTGCAGCTGCAGGTCGACCT | |
| CGAGATCAAACGGGCGGCCGCAGAACAAAAACTCATCTCAGAAGAGGATCTGAATGGGGC | |
| CGCATAGACTGTTGAAAGTTGTTTAGCAAAACCTCATACAGAAAATTCATTTACTAACGT | |
| CTGGAAAGACGACAAAACTTTAGATCGTTACGCTAACTATGAGGGCTGTCTGTGGAATGC | |
| TACAGGCGTTGTGGTTTGTACTGGTGACGAAACTCAGTGTTACGGTACATGGGTTCCTAT | |
| TGGGCTTGCTATCCCTGAAAATGAGGGTGGTGGCTCTGAGGGTGGCGGTTCTGAGGGTGG | |
| CGGTTCTGAGGGTGGCGGTACTAAACCTCCTGAGTACGGTGATACACCTATTCCGGGCTA | |
| TACTTATATCAACCCTCTCGACGGCACTTATCCGCCTGGTACTGAGCAAAACCCCGCTAA | |
| TCCTAATCCTTCTCTTGAGGAGTCTCAGCCTCTTAATACTTTCATGTTTCAGAATAATAG | |
| GTTCCGAAATAGGCAGGGTGCATTAACTGTTTATACGGGCACTGTTACTCAAGGCACTGA | |
| CCCCGTTAAAACTTATTACCAGTACACTCCTGTATCATCAAAAGCCATGTATGACGCTTA | |
| CTGGAACGGTAAATTCAGAGACTGCGCTTTCCATTCTGGCTTTAATGAGGATCCATTCGT | |
| TTGTGAATATCAAGGCCAATCGTCTGACCTGCCTCAACCTCCTGTCAATGCTGGCGGCGG | |
| CTCTGGTGGTGGTTCTGGTGGCGGCTCTGAGGGTGGCGGCTCTGAGGGTGGCGGTTCTGA | |
| GGGTGGCGGCTCTGAGGGTGGCGGTTCCGGTGGCGGCTCCGGTTCCGGTGATTTTGATTA | |
| TGAAAAAATGGCAAACGCTAATAAGGGGGCTATGACCGAAAATGCCGATGAAAACGCGCT | |
| ACAGTCTGACGCTAAAGGCAAACTTGATTCTGTCGCTACTGATTACGGTGCTGCTATCGA | |
| TGGTTTCATTGGTGACGTTTCCGGCCTTGCTAATGGTAATGGTGCTACTGGTGATTTTGC | |
| TGGCTCTAATTCCCAAATGGCTCAAGTCGGTGACGGTGATAATTCACCTTTAATGAATAA | |
| TTTCCGTCAATATTTACCTTCTTTGCCTCAGTCGGTTGAATGTCGCCCTTATGTCTTTGG | |
| CGCTGGTAAACCATATGAATTTTCTATTGATTGTGACAAAATAAACTTATTCCGTGGTGT | |
| CTTTGCGTTTCTTTTATATGTTGCCACCTTTATGTATGTATTTTCGACGTTTGCTAACAT | |
| ACTGCGTAATAAGGAGTCTTAATAAGAATTCACTGGCCGTCGTTTTACAACGTCGTGACT | |
| GGGAAAACCCTGGCGTTACCCAACTTAATCGCCTTGCAGCACATCCCCCTTTCGCCAGCT | |
| GGCGTAATAGCGAAGAGGCCCGCACCGATCGCCCTTCCCAACAGTTGCGCAGCCTGAATG | |
| GCGAATGGCGCCTGATGCGGTATTTTCTCCTTACGCATCTGTGCGGTATTTCACACCGCA | |
| TACGTCAAAGCAACCATAGTACGCGCCCTGTAGCGGCGCATTAAGCGCGGCGGGTGTGGT | |
| GGTTACGCGCAGCGTGACCGCTACACTTGCCAGCGCCCTAGCGCCCGCTCCTTTCGCTTT | |
| CTTCCCTTCCTTTCTCGCCACGTTCGCCGGCTTTCCCCGTCAAGCTCTAAATCGGGGGCT | |
| CCCTTTAGGGTTCCGATTTAGTGCTTTACGGCACCTCGACCCCAAAAAACTTGATTTGGG | |
| TGATGGTTCACGTAGTGGGCCATCGCCCTGATAGACGGTTTTTCGCCCTTTGACGTTGGA | |
| GTCCACGTTCTTTAATAGTGGACTCTTGTTCCAAACTGGAACAACACTCAACCCTATCTC | |
| GGGCTATTCTTTTGATTTATAAGGGATTTTGCCGATTTCGGCCTATTGGTTAAAAAATGA | |
| GCTGATTTAACAAAAATTTAACGCGAATTTTAACAAAATATTAACGTTTACAATTTTATG | |
| GTGCACTCTCAGTACAATCTGCTCTGATGCCGCATAGTTAAGCCAGCCCCGACACCCGCC | |
| AACACCCGCTGACGCGCCCTGACGGGCTTGTCTGCTCCCGGCATCCGCTTACAGACAAGC | |
| TGTGACCGTCTCCGGGAGCTGCATGTGTCAGAGGTTTTCACCGTCATCACCGAAACGCGC | |
| GAGACGAAAGGGCCTCGTGATACGCCTATTTTTATAGGTTAATGTCATGATAATAATGGT | |
| TTCTTAGACGTCAGGTGGCACTTTTCGGGGAAATGTGCGCGGAACCCCTATTTGTTTATT | |
| TTTCTAAATACATTCAAATATGTATCCGCTCATGAGACAATAACCCTGATAAATGCTTCA | |
| ATAATATTGAAAAAGGAAGAGTATGAGTATTCAACATTTCCGTGTCGCCCTTATTCCCTT | |
| TTTTGCGGCATTTTGCCTTCCTGTTTTTGCTCACCCAGAAACGCTGGTGAAAGTAAAAGA | |
| TGCTGAAGATCAGTTGGGTGCACGAGTGGGTTACATCGAACTGGATCTCAACAGCGGTAA | |
| GATCCTTGAGAGTTTTCGCCCCGAAGAACGTTTTCCAATGATGAGCACTTTTAAAGTTCT | |
| GCTATGTGGCGCGGTATTATCCCGTATTGACGCCGGGCAAGAGCAACTCGGTCGCCGCAT | |
| ACACTATTCTCAGAATGACTTGGTTGAGTACTCACCAGTCACAGAAAAGCATCTTACGGA | |
| TGGCATGACAGTAAGAGAATTATGCAGTGCTGCCATAACCATGAGTGATAACACTGCGGC | |
| CAACTTACTTCTGACAACGATCGGAGGACCGAAGGAGCTAACCGCTTTTTTGCACAACAT | |
| GGGGGATCATGTAACTCGCCTTGATCGTTGGGAACCGGAGCTGAATGAAGCCATACCAAA | |
| CGACGAGCGTGACACCACGATGCCTGTAGCAATGGCAACAACGTTGCGCAAACTATTAAC | |
| TGGCGAACTACTTACTCTAGCTTCCCGGCAACAATTAATAGACTGGATGGAGGCGGATAA | |
| AGTTGCAGGACCACTTCTGCGCTCGGCCCTTCCGGCTGGCTGGTTTATTGCTGATAAATC | |
| TGGAGCCGGTGAGCGTGGGTCTCGCGGTATCATTGCAGCACTGGGGCCAGATGGTAAGCC | |
| CTCCCGTATCGTAGTTATCTACACGACGGGGAGTCAGGCAACTATGGATGAACGAAATAG | |
| ACAGATCGCTGAGATAGGTGCCTCACTGATTAAGCATTGGTAACTGTCAGACCAAGTTTA | |
| CTCATATATACTTTAGATTGATTTAAAACTTCATTTTTAATTTAAAAGGATCTAGGTGAA | |
| GATCCTTTTTGATAATCTCATGACCAAAATCCCTTAACGTGAGTTTTCGTTCCACTGAGC | |
| GTCAGACCCCGTAGAAAAGATCAAAGGATCTTCTTGAGATCCTTTTTTTCTGCGCGTAAT | |
| CTGCTGCTTGCAAACAAAAAAACCACCGCTACCAGCGGTGGTTTGTTTGCCGGATCAAGA | |
| GCTACCAACTCTTTTTCCGAAGGTAACTGGCTTCAGCAGAGCGCAGATACCAAATACTGT | |
| CCTTCTAGTGTAGCCGTAGTTAGGCCACCACTTCAAGAACTCTGTAGCACCGCCTACATA | |
| CCTCGCTCTGCTAATCCTGTTACCAGTGGCTGCTGCCAGTGGCGATAAGTCGTGTCTTAC | |
| CGGGTTGGACTCAAGACGATAGTTACCGGATAAGGCGCAGCGGTCGGGCTGAACGGGGGG | |
| TTCGTGCACACAGCCCAGCTTGGAGCGAACGACCTACACCGAACTGAGATACCTACAGCG | |
| TGAGCTATGAGAAAGCGCCACGCTTCCCGAAGGGAGAAAGGCGGACAGGTATCCGGTAAG | |
| CGGCAGGGTCGGAACAGGAGAGCGCACGAGGGAGCTTCCAGGGGGAAACGCCTGGTATCT | |
| TTATAGTCCTGTCGGGTTTCGCCACCTCTGACTTGAGCGTCGATTTTTGTGATGCTCGTC | |
| AGGGGGGCGGAGCCTATGGAAAAACGCCAGCAACGCGGCCTTTTTACGGTTCCTGGCCTT | |
| TTGCTGGCCTTTTGCTCACATGTTCTTTCCTGCGTTATCCCCTGATTCTGTGGATAACCG | |
| TATTACCGCCTTTGAGTGAGCTGATACCGCTCGCCGCAGCCGAACGACCGAGCGCAGCGA | |
| GTCAGTGAGCGAGGAAGCGGAAG 4523: | |

1.5.4:
Name of vector: pTIM1

Alteration from pHEN1: The multiple cloning site, which precedes c-myc-tag, amber codon and gene 3, has been altered: It is 67 base pair longer and some of its RE sites are different. This sequence is the same as for 1.5.3 [SEQ. ID. NO. 7], except that bases 237-1648 were replaced with following sequence [SEQ. ID. NO. 8]:

CTTGCATGCAAATTCTATTTCAAGGAGACAGTCATAATGAAATACCTATTGCCTACGG
CAGCCGCTGGATTGTTATTACTCGCGGCCCAGCCGGCCATGGCCCAGGTGCAGCTGCAGG
TCACCGTCTCGAGTGGTGGAGGCGGTTCAGGCGGAGGTGGCTCTGGCGGTGGCGGATCGG
ATATCGAGCTCACTGAGATCAAACGGGCGGCCGCAGAACAAAAACTCATCTCAGAAGAGG
ATCTGAATGGGGCCGCATAGACTGTTGAAAGTTGTTTAGCAAAACCTCATACAGAAAATT
CATTTACTAACGTCTGGAAAGACGACAAAACTTTAGATCGTTACGCTAACTATGAGGGCT
GTCTGTGGAATGCTACAGGCGTTGTGGTTTGTACTGGTGACGAAACTCAGTGTTACGGTA
CATGGGTTCCTATTGGGCTTGCTATCCCTGAAAATGAGGGTGGTGGCTCTGAGGGTGGCG
GTTCTGAGGGTGGCGGTTCTGAGGGTGGCGGTACTAAACCTCCTGAGTACGGTGATACAC
CTATTCCGGGCTATACTTATATCAACCCTCTCGACGGCACTTATCCGCCTGGTACTGAGC
AAAACCCCGCTAATCCTAATCCTTCTCTTGAGGAGTCTCAGCCTCTTAATACTTTCATGT
TTCAGAATAATAGGTTCCGAAATAGGCAGGGTGCATTAACTGTTTATACGGGCACTGTTA
CTCAAGGCACTGACCCCGTTAAAACTTATTACCAGTACACTCCTGTATCATCAAAAGCCA
TGTATGACGCTTACTGGAACGGTAAATTCAGAGACTGCGCTTTCCATTCTGGCTTTAATG
AGGATCCATTCGTTTGTGAATATCAAGGCCAATCGTCTGACCTGCCTCAACCTCCTGTCA
ATGCTGGCGGCGGCTCTGGTGGTGGTTCTGGTGGCGGCTCTGAGGGTGGCGGCTCTGAGG
GTGGCGGTTCTGAGGGTGGCGGCTCTGAGGGTGGCGGTTCCGGTGGCGGCTCCGGTTCCG
GTGATTTTGATTATGAAAAAATGGCAAACGCTAATAAGGGGGCTATGACCGAAAATGCCG
ATGAAAACGCGCTACAGTCTGACGCTAAAGGCAAACTTGATTCTGTCGCTACTGATTACG
GTGCTGCTATCGATGGTTTCATTGGTGACGTTTCCGGCCTTGCTAATGGTAATGGTGCTA
CTGGTGATTTTGCTGGCTCTAATTCCCAAATGGCTCAAGTCGGTGACGGTGATAATTCAC
CTTTAATGAATAATTTCCGTCAATATTTACCTTCTTTGCCTCAGTCGGTTGAATGTCGCC
CTTATGTCTTTGGCGCTGGTAAACCATATGAATTTTCTATTGATTGTGACAAAATAAACT
TATTCCGTGGTGTCTTTGCGTTTCTTTTATATGTTGCCACCTTTATGTATGTATTTTCGA
CGTTTGCTAACATACTGCGTAATAAGGAGTCTTAATAAGAA.

1.5.5:
Name of vector: pMAB2
Alteration from pTIM-1: Alterations at c-myc-tag/g3 junction: Replace amber stop codon with alanine-codon.

Same as for 1.5.3 [SEQ. ID. NO. 7], except that bases 237-1648 were replaced with following sequence [SEQ. ID. NO. 9]:

CTTGCATGCAAATTCTATTTCAGGAGACAGTCATAATGAAATACCTATTGCCTACGG
CAGCCGCTGGATTGTTATTACTCGCGGCCCAGCCGGCCATGGCCCAGGTGCAGCTGCAGG
TCACCGTCTCGAGTGGTGGAGGCGGTTCAGGCGGAGGTGGCTCTGGCGGTGGCGGATCGG
ATATCGAGCTCACTGAGATCAAACGGGCGGCCGCAGAACAAAAACTCATCTCAGAAGAGG
ATCTAAATGGGGCTGCAGCGACTGTTGAAAGTTGTTTAGCAAAACCTCATACAGAAAATT
CATTTACTAACGTCTGGAAAGACGACAAAACTTTAGATCGTTACGCTAACTATGAGGGCT
GTCTGTGGAATGCTACGGGCGTTGTGGTTTGCACTGGTGACGAAACTCAGTGTTACGGTA
CATGGGTTCCTATTGGGCTTGCTATCCCTGAAATGAGGGTGGTGGCTCTGAGGGTGGCGG
GTTCTGAGGGTGGCGGTTCTGAGGGTGGCGGTACTAAACCTCCAGAGTACGGTGATACAC
CTATTCCGGGCTATACTTATATCAACCCTCTCGACGGCACTTATCCGCCTGGTACTGAGC
AAAACCCCGCTAATCCTAATCCTTCTCTTGAGGAGTCTCAGCCTCTTAATACTTTCATGT
TTCAGAATAATAGGTTCCGAAATAGGCAGGGTGCATTAACTGTTTATACGGGCACTGTTA
CTCAAGGCACTGACCCCGTTAAAACTTATTACCAGTACACTCCTGTATCATCAAAAGCCA
TGTATGACGCTTACTGGAACGGTAAATTCAGAGACTGCGCTTTCCATTCTGGCTTTAATG
AGGATCCATTCGTTTGTGAATATCAAGGCCAATCGTCTGACCTGCCTCAACCTCCTGTCA
ATGCTGGCGGCGGCTCTGGTGGTGGTTCTGGTGGCGGCTCTGAGGGTGGCGGCTCTGAGG
GTGGCGGTTCTGAGGGTGGCGGCTCTGAGGGTGGCGGTTCCGGTGGCGGCTCCGGTTCCG
GTGATTTTGATTATGAAAAAATGGCAAACGCTAATAAGGGGGCTATGACCGAAAATGCCG
ATGAAAACGCGCTACAGTCTGACGCTAAAGGCAAACTTGATTCTGTCGCTACTGATTACG
GTGCTGCTATCGATGGTTTCATTGGTGACGTTTCCGGCCTTGCTAATGGTAATGGTGCTA
CTGGTGATTTTGCTGGCTCTAATTCCCAAATGGCTCAAGTCGGTGACGGTGATAATTCAC
CTTTAATGAATAATTTCCGTCAATATTTACCTTCTTTGCCTCAGTCGGTTGAATGTCGCC
CTTATGTCTTTGGCGCTGGTAAACCATATGAATTTTCTATTGATTGTGACAAAATAAACT
TATTCCGTGGTGTCTTTGCGTTTCTTTTATATGTTGCCACCTTTATGTATGTATTTTCGA
CGTTTGCTAACATACTGCGTAATAAGGAGTCTTAATAAGAA

1.5.6A:
Name of vector: pMAB3
Alteration from pMAB-2: Rendering g3 locus bicistronic, to allow for expression of antibody Fab fragments: A second RBS-sequence+pelB leader was inserted (—same amino acid sequence as before, different DNA sequence). Both pelB-leaders followed by unique RE sites to allow for cloning of separate Fab genes (i.e. V_{L}C_{L} and V_{H}C_{H})

Same as for 1.5.3 [SEQ. ID. NO. 7], except that bases 237-1648 were replaced with following sequence [SEQ. ID. NO. 10]:

CTTGCATGCAAATTCTATTTCAAGGAGACAGTCATAATGAAATACCTATTGCCTACGG
CAGCCGCTGGATTGTTATTACTCGCGGCCCAGCCGGCCATGGCTCTAGAGTAAGGAGGCA
GTCATAATGAAGTACCTTTTGCCAACGGCTGCCGCTGGCTTGTTATTGCTCGCGGCACAG
CCGGCAATTGCCTCGAGTGGTGGAGGCGGTTCAGGCGGAGGTGGCTCTGGCGGTGGCGGA
TCGGATATCGAGCTCACTGAGATCAAACGGGCGGCCGCAGAACAAAAACTCATCTCAGAA
GAGGATCTAAATGGGGCTGCAGCGACTGTTGAAAGTTGTTTAGCAAAACCTCATACAGAA
AATTCATTTACTAACGTCTGGAAAGACGACAAAACTTTAGATCGTTACGCTAACTATGAG
GGCTGTCTGTGGAATGCTACGGGCGTTGTGGTTTGCACTGGTGACGAAACTCAGTGTTAC
GGTACATGGGTTCCTATTGGGCTTGCTATCCCTGAAAATGAGGGTGGTGGCTCTGAGGGT
GGCGGTTCTGAGGGTGGCGGTTCTGAGGGTGGCGGTACTAAACCTCCAGAGTACGGTGAT
ACACCTATTCCGGGCTATACTTATATCAACCCTCTCGACGGCACTTATCCGCCTGGTACT
GAGCAAAACCCCGCTAATCCTAATCCTTCTCTTGAGGAGTCTCAGCCTCTTAATACTTTC
ATGTTTCAGAATAATAGGTTCCGAAATAGGCAGGGTGCATTAACTGTTTATACGGGCACT
GTTACTCAAGGCACTGACCCCGTTAAAACTTATTACCAGTACACTCCTGTATCATCAAAA
GCCATGTATGACGCTTACTGGAACGGTAAATTCAGAGACTGCGCTTTCCATTCTGGCTTT
AATGAGGATCCATTCGTTTGTGAATATCAAGGCCAATCGTCTGACCTGCCTCAACCTCCT
GTCAATGCTGGCGGCGGCTCTGGTGGTGGTTCTGGTGGCGGCTCTGAGGGTGGCGGCTCT
GAGGGTGGCGGTTCTGAGGGTGGCGGCTCTGAGGGTGGCGGTTCCGGTGGCGGCTCCGGT
TCCGGTGATTTTGATTATGAAAAAATGGCAAACGCTAATAAGGGGGCTATGACCGAAAAT
GCCGATGAAAACGCGCTACAGTCTGACGCTAAAGGCAAACTTGATTCTGTCGCTACTGAT
TACGGTGCTGCTATCGATGGTTTCATTGGTGACGTTTCCGGCCTTGCTAATGGTAATGGT
GCTACTGGTGATTTTGCTGGCTCTAATTCCCAAATGGCTCAAGTCGGTGACGGTGATAAT
TCACCTTTAATGAATAATTTCCGTCAATATTTACCTTCTTTGCCTCAGTCGGTTGAATGT
CGCCCTTATGTCTTTGGCGCTGGTAAACCATATGAATTTTCTATTGATTGTGACAAAATA
AACTTATTCCGTGGTGTCTTTGCGTTTCTTTTATATGTTGCCACCTTTATGTATGTATTT
TCGACGTTTGCTAACATACTGCGTAATAAGGAGTCTTAATAAGAA

1.5.6B:
Name of vector: pMAB29
Alteration from pMAB3: Insertion of Fab genes, encoding for a fully human, anti-tetanus toxoid antibody fragment.

Same as for 1.5.3 [SEQ. ID. NO. 7], except that bases 237-1648 were replaced with following sequence [SEQ. ID. NO. 11]:

CTTGCATGCAAATTCTATTTCAAGGAGACAGTCATAATGAAATACCTATTGCCTACGG
CAGCCGCTGGATTGTTATTACTCGCGGCCCAGCCGGCCATGGCTGACATCCAGATGACCC
AGTCTCCATCCTCCCTGTCTGCATCTGTAGGAGACAGAGTCATCATCACTTGCCGGGCAA
GTCAGAGTATTAGCACCTATTTAAATTGGTATCAGCAGAAACCAGGGAAAGCCCCTAAAC
TCCTGATCTATTATGCAACCAATTTGCAAAGTGGGGTCCCATCAAGGTTCAGTGGCAGTG
GATCTGGGACAGATTTCACTCTCACCATCAGCAGTCTGCAACCTGAAGATTTTGCGACTT
ATTATTGTCAACAGAGTTCCAACACCGTCACTTTCGGCCCTGGGACCAAAGTGGATATGA
AGCGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGG
ACAGCAAGGACAGCACCTACAGCCTCAGCAGCACCCTGACGCTGAGCAAAGCAGACTACG
AGAAACACAAAGTCTACGCCTGCGAAGTCACCCATCAGGGCCTGAGTTCGCCCGTCACAA
AGAGCTTCAACAGGGGAGAGTGTTAATTCTAGAGTAAGGAGGCAGTCATAATGAAGTACC
TTTTGCCAACGGCTGCCGCTGGCTTGTTATTGCTCGCGGCACAGCCGGCAATTGCCCAGG
TGCAGCTGGTGCAGTCTGGGGGAGGCTTGGTACAGCCTGGGGGGTCCGTGAGACTCTCCT
GTGCAGCCTCTGGATTCAGTTTTAGCAGCTATGCCATGAGCTGGGTCCGCCAGGCTCCAG
GGATGGGGCTGGAGTGGGTCGCGGCTATTAGTGCTAGAGGAACTACCACATATTATGCAG
ACTCCGTGACGGGCCGATTGACCATCTCCAGAGACAATTCCATGAACACGCTATATCTGC
ACTTGAACAGCCTGAGAGCCGAGGACACGGCCGTTTATTACTGTGCGAAAGCGGGAAAAC
AGTGGCTGGCCCACTACTACTTTGACTCCTGGGGCCAGGGAACCCTGGTCACCGTCTCCT
CAGCCTCCACCAAGGGCCCATCGGTCTTCCCCCTGGCACCCTCCTCCAAGAGCACCTCTG
GGGGCACAGCGGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGA
CCTACATCTGCAACGTGAATCACAAGCCCAGCAACACCAAGGTGGACAAGAAAGTTGAGC
CCAAATCTTGTGACAAAGCGGCCGCAGAACAAAAACTCATCTCAGAAGAGGATCTAAATG
GGGCTGCAGCGACTGTTGAAAGTTGTTTAGCAAAACCTCATACAGAAAATTCATTTACTA
ACGTCTGGAAAGACGACAAAACTTTAGATCGTTACGCTAACTATGAGGGCTGTCTGTGGA
ATGCTACGGGCGTTGTGGTTTGCACTGGTGACGAAACTCAGTGTTACGGTACATGGGTTC
CTATTGGGCTTGCTATCCCTGAAAATGAGGGTGGTGGCTCTGAGGGTGGCGGTTCTGAGG
GTGGCGGTTCTGAGGGTGGCGGTACTAAACCTCCAGAGTACGGTGATACACCTATTCCGG
GCTATACTTATATCAACCCTCTCGACGGCACTTATCCGCCTGGTACTGAGCAAAACCCCG
CTAATCCTAATCCTTCTCTTGAGGAGTCTCAGCCTCTTAATACTTTCATGTTTCAGAATA
ATAGGTTCCGAAATAGGCAGGGTGCATTAACTGTTTATACGGGCACTGTTACTCAAGGCA
CTGACCCCGTTAAAACTTATTACCAGTACACTCCTGTATCATCAAAAGCCATGTATGACG
CTTACTGGAACGGTAAATTCAGAGACTGCGCTTTCCATTCTGGCTTTAATGAGGATCCAT
TCGTTTGTGAATATCAAGGCCAATCGTCTGACCTGCCTCAACCTCCTGTCAATGCTGGCG
GCGGCTCTGGTGGTGGTTCTGGTGGCGGCTCTGAGGGTGGCGGCTCTGAGGGTGGCGGTT
CTGAGGGTGGCGGCTCTGAGGGTGGCGGTTCCGGTGGCGGCTCCGGTTCCGGTGATTTTG
ATTATGAAAAAATGGCAAACGCTAATAAGGGGGCTATGACCGAAAATGCCGATGAAAACG
CGCTACAGTCTGACGCTAAAGGCAAACTTGATTCTGTCGCTACTGATTACGGTGCTGCTA
TCGATGGTTTCATTGGTGACGTTTCCGGCCTTGCTAATGGTAATGGTGCTACTGGTGATT
TTGCTGGCTCTAATTCCCAAATGGCTCAAGTCGGTGACGGTGATAATTCACCTTTAATGA
ATAATTTCCGTCAATATTTACCTTCTTTGCCTCAGTCGGTTGAATGTCGCCCTTATGTCT
TTGGCGCTGGTAAACCATATGAATTTTCTATTGATTGTGACAAAATAAACTTATTCCGTG
GTGTCTTTGCGTTTCTTTTATATGTTGCCACCTTTATGTATGTATTTTCGACGTTTGCTA
ACATACTGCGTAATAAGGAGTCTTAATAAGAA

1.5.7.A.1
Name of vector: pMAB65
Alteration from pMAB3: Replacing structural g3 and sequence immediately downstream (but not upstream, bicistronic region). Insertion-replacement cloning event changed several features of g3: Inserted NheI-site 5′ of structural g3; c-myc tag existing 5′ of structural g3 was deleted; Full-length g3 replaced by a shorter version, having a N-terminal truncation; Another NheI site and a His₆-encoding sequence were inserted 3′ of g3—together with the 5′ NheI this allows for switching to expression of soluble His₆-tagged Fab fragment; Also, a transcriptional stop was added 3′ of these elements.

Same as for 1.5.3 [SEQ. ID. NO. 7], except that bases 237-1648 were replaced with following sequence [SEQ. ID. NO. 12]:

CTTGCATGCAATTCTATTTCAGGAGACAGTCATAATGAAATACCTATTGCCTACGG
CAGCCGCTGGATTGTTATTACTCGCGGCCCAGCCGGCCATGGCTCTAGAGTAAGGAGGCA
GTCATAATGAAGTACCTTTTGCCAACGGCTGCCGCTGGCTTGTTATTGCTCGCGGCACAG
CCGGCAATTGCCTCGAGTGGTGGAGGCGGTTCAGGCGGAGGTGGCTCTGGCGGTGGCGGA
TCGGATATCGAGCTCACTGAGATCAAACGGGCGGCCGCTAGCCCTCAACCTCCTGTCAAT
GCTGGCGGCGGCTCTGGTGGTGGTTCTGGTGGCGGCTCTGAGGGTGGCGGCTCTGAGGGT
GGCGGTTCTGAGGGTGGCGGCTCTGAGGGTGGCGGTTCCGGTGGCGGCTCCGGTTCCGGT
GATTTTGATTATGAAAAAATGGCAAACGCTAATAAGGGGGCTATGACCGAAAATGCCGAT
GAAAACGCGCTACAGTCTGACGCTAAAGGCAAACTTGATTCTGTCGCTACTGATTACGGT
GCTGCTATCGATGGTTTCATTGGTGACGTTTCCGGCCTTGCTAATGGTAATGGTGCTACT
GGTGATTTTGCTGGCTCTAATTCCCAAATGGCTCAAGTCGGTGACGGTGATAATTCACCT
TTAATGAATAATTTCCGTCAATATTTACCTTCTTTGCCTCAGTCGGTTGAATGTCGCCCT
TATGTCTTTGGCGCTGGTAAACCATATGAATTTTCTATTGATTGTGACAAAATAAACTTA
TTCCGTGGTGTCTTTGCGTTTCTTTTATATGTTGCCACCTTTATGTATGTATTTTCGACG
TTTGCTAACATACTGCGTAATAAGGAGTCTTAATAAGCTAGCCATCACCACCATCATCAC
TAATAATGAAAGCCCGCCTAATGAGCGGGCTTTTTTTTGAA

1.5.7.A.2
Name of vector: pMAB66
Alteration from pMAB65: Insertion of anti-TT specific Fab fragments

Same as for 1.5.3 [SEQ. ID. NO. 7], except that bases 237-1648 were replaced with following sequence [SEQ. ID. NO. 13]:

CTTGCATGCAAATTCTATTTCAAGGAGACAGTCATAATGAAATACCTATTGCCTACGGCAGC
CGCTGGATTGTTATTACTCGCGGCCCAGCCGGCCATGGCTGACATCCAGATGACCCAGTCTC
CATCCTCCCTGTCTGCATCTGTAGGAGACAGAGTCATCATCACTTGCCGGGCAAGTCAGAGT
ATTAGCACCTATTTAAATTGGTATCAGCAGAAACCAGGGAAAGCCCCTAAACTCCTGATCTA
TTATGCAACCAATTTGCAAAGTGGGGTCCCATCAAGGTTCAGTGGCAGTGGATCTGGGACAG
ATTTCACTCTCACCATCAGCAGTCTGCAACCTGAAGATTTTGCGACTTATTATTGTCAACAG
AGTTCCAACACCGTCACTTTCGGCCCTGGGACCAAAGTGGATATGAAGCGAACTGTGGCTGC
ACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCG
AAGTCACCCATCAGGGCCTGAGTTCGCCCGTCACAAAGAGCTTCAACAGGGGAGAGTGTTAA
TTCTAGAGTAAGGAGGCAGTCATAATGAAGTACCTTTTGCCAACGGCTGCCGCTGGCTTGTT
ATTGCTCGCGGCACAGCCGGCAATTGCCCAGGTGCAGCTGGTGCAGTCTGGGGGAGGCTTGG
TACAGCCTGGGGGGTCCGTGAGACTCTCCTGTGCAGCCTCTGGATTCAGTTTTAGCAGCTAT
GCCATGAGCTGGGTCCGCCAGGCTCCAGGGATGGGGCTGGAGTGGGTCGCGGCTATTAGTGC
TAGAGGAACTACCACATATTATGCAGACTCCGTGACGGGCCGATTGACCATCTCCAGAGACA
ATTCCATGAACACGCTATATCTGCACTTGAACAGCCTGAGAGCCGAGGACACGGCCGTTTAT
TACTGTGCGAAAGCGGGAAAACAGTGGCTGGCCCACTACTACTTTGACTCCTGGGGCCAGGG
AACCCTGGTCACCGTCTCCTCAGCCTCCACCAAGGGCCCATCGGTCTTCCCCCTGGCACCCT
CCTCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCT
TGGGCACCCAGACCTACATCTGCAACGTGAATCACAAGCCCAGCAACACCAAGGTGGACAAG
AAAGTTGAGCCCAAATCTTGTGACAAAGCGGCCGCTAGCCCTCAACCTCCTGTCAATGCTGG
CGGCGGCTCTGGTGGTGGTTCTGGTGGCGGCTCTGAGGGTGGCGGCTCTGAGGGTGGCGGTT
CTGAGGGTGGCGGCTCTGAGGGTGGCGGTTCCGGTGGCGGCTCCGGTTCCGGTGATTTTGAT
TATGAAAAAATGGCAAACGCTAATAAGGGGGCTATGACCGAAAATGCCGATGAAAACGCGCT
ACAGTCTGACGCTAAAGGCAAACTTGATTCTGTCGCTACTGATTACGGTGCTGCTATCGATG
GTTTCATTGGTGACGTTTCCGGCCTTGCTAATGGTAATGGTGCTACTGGTGATTTTGCTGGC
TCTAATTCCCAAATGGCTCAAGTCGGTGACGGTGATAATTCACCTTTAATGAATAATTTCCG
TCAATATTTACCTTCTTTGCCTCAGTCGGTTGAATGTCGCCCTTATGTCTTTGGCGCTGGTA
AACCATATGAATTTTCTATTGATTGTGACAAAATAAACTTATTCCGTGGTGTCTTTGCGTTT
CTTTTATATGTTGCCACCTTTATGTATGTATTTTCGACGTTTGCTAACATACTGCGTAATAA
GGAGTCTTAATAAGCTAGCCATCACCACCATCATCACTAATAATGAAAGCCCGCCTAATGAG
CGGGCTTTTTTTTGAA

1.5.7.B.1
Name of vector: pMAB64
Alteration from pMAB3: pMAB3 was modified exactly the same way as in “1.5.7.A.1”, except that g3 was not truncated.

Same as for 1.5.3[SEQ. ID. NO. 7], except that bases 237-1648 were replaced with following sequence [SEQ. ID. NO. 14]:

CTTGCATGCAAATTCTATTTCAAGGAGACAGTCATAATGAAATACCTATTGCCTACGGCAGC
CGCTGGATTGTTATTACTCGCGGCCCAGCCGGCCATGGCTCTAGAGTAAGGAGGCAGTCATA
ATGAAGTACCTTTTGCCAACGGCTGCCGCTGGCTTGTTATTGCTCGCGGCACAGCCGGCAAT
TGCCTCGAGTGGTGGAGGCGGTTCAGGCGGAGGTGGCTCTGGCGGTGGCGGATCGGATATCG
AGCTCACTGAGATCAAACGGGCGGCCGCTAGCACTGTTGAAAGTTGTTTAGCAAAACCTCAT
ACAGAAAATTCATTTACTAACGTCTGGAAAGACGACAAAACTTTAGATCGTTACGCTAACTA
TGAGGGCTGTCTGTGGAATGCTACGGGCGTTGTGGTTTGCACTGGTGACGAAACTCAGTGTT
ACGGTACATGGGTTCCTATTGGGCTTGCTATCCCTGAAAATGAGGGTGGTGGCTCTGAGGGT
GGCGGTTCTGAGGGTGGCGGTTCTGAGGGTGGCGGTACTAAACCTCCAGAGTACGGTGATAC
ACCTATTCCGGGCTATACTTATATCAACCCTCTCGACGGCACTTATCCGCCTGGTACTGAGC
AAAACCCCGCTAATCCTAATCCTTCTCTTGAGGAGTCTCAGCCTCTTAATACTTTCATGTTT
CAGAATAATAGGTTCCGAAATAGGCAGGGTGCATTAACTGTTTATACGGGCACTGTTACTCA
AGGCACTGACCCCGTTAAAACTTATTACCAGTACACTCCTGTATCATCAAAAGCCATGTATG
ACGCTTACTGGAACGGTAAATTCAGAGACTGCGCTTTCCATTCTGGCTTTAATGAGGATCCA
TTCGTTTGTGAATATCAAGGCCAATCGTCTGACCTGCCTCAACCTCCTGTCAATGCTGGCGG
CGGCTCTGGTGGTGGTTCTGGTGGCGGCTCTGAGGGTGGCGGCTCTGAGGGTGGCGGTTCTG
AGGGTGGCGGCTCTGAGGGTGGCGGTTCCGGTGGCGGCTCCGGTTCCGGTGATTTTGATTAT
GAAAAAATGGCAAACGCTAATAAGGGGGCTATGACCGAAAATGCCGATGAAAACGCGCTACA
GTCT
GACGCTAAAGGCAAACTTGATTCTGTCGCTACTGATTACGGTGCTGCTATCGATGGTTTCAT
TGGTGACGTTTCCGGCCTTGCTAATGGTAATGGTGCTACTGGTGATTTTGCTGGCTCTAATT
CCCAAATGGCTCAAGTCGGTGACGGTGATAATTCACCTTTAATGAATAATTTCCGTCAATAT
TTACCTTCTTTGCCTCAGTCGGTTGAATGTCGCCCTTATGTCTTTGGCGCTGGTAAACCATA
TGAATTTTCTATTGATTGTGACAAAATAAACTTATTCCGTGGTGTCTTTGCGTTTCTTTTAT
ATGTTGCCACCTTTATGTATGTATTTTCGACGTTTGCTAACATACTGCGTAATAAGGAGTCT
TAATAAGCTAGCCATCACCACCATCATCACTAATAATGAAAGCCCGCCTAATGAGCGGGCTT
TTTTTTGAA

1.5.7.B.2
Name of vector: pMAB77
Alteration from pMAB64: Insertion of anti-TT specific Fab fragments

Same as for 1.5.3 [SEQ. ID. NO. 7] except that bases 237-1648 were replaced with following sequence [SEQ. ID. NO. 15]

CTTGCATGCAAATTCTATTTCAAGGAGACAGTCATAATGAAATACCTATTGCCTACGGCAGC
CGCTGGATTGTTATTACTCGCGGCCCAGCCGGCCATGGCTGACATCCAGATGACCCAGTCTC
CATCCTCCCTGTCTGCATCTGTAGGAGACAGAGTCATCATCACTTGCCGGGCAAGTCAGAGT
ATTAGCACCTATTTAAATTGGTATCAGCAGAAACCAGGGAAAGCCCCTAAACTCCTGATCTA
TTATGCAACCAATTTGCAAAGTGGGGTCCCATCAAGGTTCAGTGGCAGTGGATCTGGGACAG
ATTTCACTCTCACCATCAGCAGTCTGCAACCTGAAGATTTTGCGACTTATTATTGTCAACAG
AGTTCCAACACCGTCACTTTCGGCCCTGGGACCAAAGTGGATATGAAGCGAACTGTGGCTGC
ACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCG
AAGTCACCCATCAGGGCCTGAGTTCGCCCGTCACAAAGAGCTTCAACAGGGGAGAGTGTTAA
TTCTAGAGTAAGGAGGCAGTCATAATGAAGTACCTTTTGCCAACGGCTGCCGCTGGCTTGTT
ATTGCTCGCGGCACAGCCGGCAATTGCCCAGGTGCAGCTGGTGCAGTCTGGGGGAGGCTTGG
TACAGCCTGGGGGGTCCGTGAGACTCTCCTGTGCAGCCTCTGGATTCAGTTTTAGCAGCTAT
GCCATGAGCTGGGTCCGCCAGGCTCCAGGGATGGGGCTGGAGTGGGTCGCGGCTATTAGTGC
TAGAGGAACTACCACATATTATGCAGACTCCGTGACGGGCCGATTGACCATCTCCAGAGACA
ATTCCATGAACACGCTATATCTGCACTTGAACAGCCTGAGAGCCGAGGACACGGCCGTTTAT
TACTGTGCGAAAGCGGGAAAACAGTGGCTGGCCCACTACTACTTTGACTCCTGGGGCCAGGG
AACCCTGGTCACCGTCTCCTCAGCCTCCACCAAGGGCCCATCGGTCTTCCCCCTGGCACCCT
CCTCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCT
TGGGCACCCAGACCTACATCTGCAACGTGAATCACAAGCCCAGCAACACCAAGGTGGACAAG
AAAGTTGAGCCCAAATCTTGTGACAAAGCGGCCGCTAGCACTGTTGAAAGTTGTTTAGCAAA
ACCTCATACAGAAAATTCATTTACTAACGTCTGGAAAGACGACAAAACTTTAGATCGTTACG
CTAACTATGAGGGCTGTCTGTGGAATGCTACGGGCGTTGTGGTTTGCACTGGTGACGAAACT
CAGTGTTACGGTACATGGGTTCCTATTGGGCTTGCTATCCCTGAAAATGAGGGTGGTGGCTC
TGAGGGTGGCGGTTCTGAGGGTGGCGGTTCTGAGGGTGGCGGTACTAAACCTCCAGAGTACG
GTGATACACCTATTCCGGGCTATACTTATATCAACCCTCTCGACGGCACTTATCCGCCTGGT
ACTGAGCAAAACCCCGCTAATCCTAATCCTTCTCTTGAGGAGTCTCAGCCTCTTAATACTTT
CATGTTTCAGAATAATAGGTTCCGAAATAGGCAGGGTGCATTAACTGTTTATACGGGCACTG
TTACTCAAGGCACTGACCCCGTTAAAACTTATTACCAGTACACTCCTGTATCATCAAAAGCC
ATGTATGACGCTTACTGGAACGGTAAATTCAGAGACTGCGCTTTCCATTCTGGCTTTAATGA
GGATCCATTCGTTTGTGAATATCAAGGCCAATCGTCTGACCTGCCTCAACCTCCTGTCAATG
CTGGCGGCGGCTCTGGTGGTGGTTCTGGTGGCGGCTCTGAGGGTGGCGGCTCTGAGGGTGGC
GGTTCTGAGGGTGGCGGCTCTGAGGGTGGCGGTTCCGGTGGCGGCTCCGGTTCCGGTGATTT
TGATTATGAAAAAATGGCAAACGCTAATAAGGGGGCTATGACCGAAAATGCCGATGAAAACG
CGCTACAGTCTGACGCTAAAGGCAAACTTGATTCTGTCGCTACTGATTACGGTGCTGCTATC
GATGGTTTCATTGGTGACGTTTCCGGCCTTGCTAATGGTAATGGTGCTACTGGTGATTTTGC
TGGCTCTAATTCCCAAATGGCTCAAGTCGGTGACGGTGATAATTCACCTTTAATGAATAATT
TCCGTCAATATTTACCTTCTTTGCCTCAGTCGGTTGAATGTCGCCCTTATGTCTTTGGCGCT
GGTAAACCATATGAATTTTCTATTGATTGTGACAAA
ATAAACTTATTCCGTGGTGTCTTTGCGTTTCTTTTATATGTTGCCACCTTTATGTATGTATT
TTCGACGTTTGCTAACATACTGCGTAATAAGGAGTCTTAATAAGCTAGCCATCACCACCATC
ATCACTAATAATGAAAGCCCGCCTAATGAGCGGGCTTTTTTTTGAA

1.5.8:
Name of vector: pMAB86
Alteration from pMAB65: Alterations of the second (3′) cloning site of the bicistronic g3-locus. Two cloning steps gave the following alterations: Insertion of a C_{H}1 region of human IgG1; Addition of extra RE sites between second pelB leader and C_{H}1 to allow for crippling of vector before inserting V_{H} genes.

Same as for 1.5.3[SEQ. ID. NO. 7] except that bases 237-1648 were replaced with following sequence [SEQ. ID. NO. 16]:

CTTGCATGCAAATTCTATTTCAAGGAGACAGTCATAATGAAATACCTATTGCCTACGG
CAGCCGCTGGATTGTTATTACTCGCGGCCCAGCCGGCCATGGCTCTAGAGTAAGGAGGCA
GTCATAATGAAGTACCTTTTGCCAACGGCTGCCGCTGGCTTGTTATTGCTCGCGGCACAG
CCGGCAATTGGGCGCGCCTAGTCGACCAAGGGCCCATCGGTCTTCCCCCTGGCACCCTCC
TCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACACCAAGGTG
GACAAGAAAGTTGAGCCCAAATCTTGTGACAAAGCGGCCGCTAGCCCTCAACCTCCTGTC
AATGCTGGCGGCGGCTCTGGTGGTGGTTCTGGTGGCGGCTCTGAGGGTGGCGGCTCTGAG
GGTGGCGGTTCTGAGGGTGGCGGCTCTGAGGGTGGCGGTTCCGGTGGCGGCTCCGGTTCC
GGTGATTTTGATTATGAAAAAATGGCAAACGCTAATAAGGGGGCTATGACCGAAAATGCC
GATGAAAACGCGCTACAGTCTGACGCTAAAGGCAAACTTGATTCTGTCGCTACTGATTAC
GGTGCTGCTATCGATGGTTTCATTGGTGACGTTTCCGGCCTTGCTAATGGTAATGGTGCT
ACTGGTGATTTTGCTGGCTCTAATTCCCAAATGGCTCAAGTCGGTGACGGTGATAATTCA
CCTTTAATGAATAATTTCCGTCAATATTTACCTTCTTTGCCTCAGTCGGTTGAATGTCGC
CCTTATGTCTTTGGCGCTGGTAAACCATATGAATTTTCTATTGATTGTGACAAAATAAAC
TTATTCCGTGGTGTCTTTGCGTTTCTTTTATATGTTGCCACCTTTATGTATGTATTTTCG
ACGTTTGCTAACATACTGCGTAATAAGGAGTCTTAATAAGCTAGCCATCACCACCATCAT
CACTAATAATGAAAGCCCGCCTAATGAGCGGGCTTTTTTTTGAA

1.5.9:
Name of vector: pMAB87
Alteration from pMAB86: Exchange of short g3 for full-length g3, derived from pMAB64. All other features of vectors kept identical.

Same as for 1.5.3 [SEQ. ID. NO. 7], except that bases 237-1648 were replaced with following sequence [SEQ. ID. NO. 17]:

CTTGCATGCAATTCTATTTCAAGGAGACAGTCATAATGAAATACCTATTGCCTACGG
CAGCCGCTGGATTGTTATTACTCGCGGCCCAGCCGGCCATGGCTCTAGAGTAAGGAGGCA
GTCATAATGAAGTACCTTTTGCCAACGGCTGCCGCTGGCTTGTTATTGCTCGCGGCACAG
CCGGCAATTGGGCGCGCCTAGTCGACCAAGGGCCCATCGGTCTTCCCCCTGGCACCCTCC
TCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACACCAAGGTG
GACAAGAAAGTTGAGCCCAAATCTTGTGACAAAGCGGCCGCTAGCACTGTTGAAAGTTGT
TTAGCAAAACCTCATACAGAAAATTCATTTACTAACGTCTGGAAAGACGACAAAACTTTA
GATCGTTACGCTAACTATGAGGGCTGTCTGTGGAATGCTACGGGCGTTGTGGTTTGCACT
GGTGACGAAACTCAGTGTTACGGTACATGGGTTCCTATTGGGCTTGCTATCCCTGAAAAT
GAGGGTGGTGGCTCTGAGGGTGGCGGTTCTGAGGGTGGCGGTTCTGAGGGTGGCGGTACT
AAACCTCCAGAGTACGGTGATACACCTATTCCGGGCTATACTTATATCAACCCTCTCGAC
GGCACTTATCCGCCTGGTACTGAGCAAAACCCCGCTAATCCTAATCCTTCTCTTGAGGAG
TCTCAGCCTCTTAATACTTTCATGTTTCAGAATAATAGGTTCCGAAATAGGCAGGGTGCA
TTAACTGTTTATACGGGCACTGTTACTCAAGGCACTGACCCCGTTAAAACTTATTACCAG
TACACTCCTGTATCATCAAAAGCCATGTATGACGCTTACTGGAACGGTAAATTCAGAGAC
TGCGCTTTCCATTCTGGCTTTAATGAGGATCCATTCGTTTGTGAATATCAAGGCCAATCG
TCTGACCTGCCTCAACCTCCTGTCAATGCTGGCGGCGGCTCTGGTGGTGGTTCTGGTGGC
GGCTCTGAGGGTGGCGGCTCTGAGGGTGGCGGTTCTGAGGGTGGCGGCTCTGAGGGTGGC
GGTTCCGGTGGCGGCTCCGGTTCCGGTGATTTTGATTATGAAAAAATGGCAAACGCTAAT
AAGGGGGCTATGACCGAAAATGCCGATGAAAACGCGCTACAGTCTGACGCTAAAGGCAAA
CTTGATTCTGTCGCTACTGATTACGGTGCTGCTATCGATGGTTTCATTGGTGACGTTTCC
GGCCTTGCTAATGGTAATGGTGCTACTGGTGATTTTGCTGGCTCTAATTCCCAAATGGCT
CAAGTCGGTGACGGTGATAATTCACCTTTAATGAATAATTTCCGTCAATATTTACCTTCT
TTGCCTCAGTCGGTTGAATGTCGCCCTTATGTCTTTGGCGCTGGTAAACCATATGAATTT
TCTATTGATTGTGACAAAATAAACTTATTCCGTGGTGTCTTTGCGTTTCTTTTATATGTT
GCCACCTTTATGTATGTATTTTCGACGTTTGCTAACATACTGCGTAATAAGGAGTCTTAA
TAAGCTAGCCATCACCACCATCATCACTAATAATGAAAGCCCGCCTAATGAGCGGGCTTT
TTTTTGAA

1.5.10:
Name of vector: pMAB93
Alteration from pMAB87: Exchange of ColE1 origin and part of Amp^{R}-gene for corresponding segments from vector pBR322.

New sequence [SEQ. ID. NO. 18]:

AGCGCCCAATACGCAAACCGCCTCTCCCCGCGCGTTGGCCGATTCATTAATGCAGCTGGCACGACAGGTTTCCCGA
CTGGAAAGCGGGCAGTGAGCGCAACGCAATTAATGTGAGTTAGCTCACTCATTAGGCACCCCAGGCTTTACACTTT
ATGCTTCCGGCTCGTATGTTGTGTGGAATTGTGAGCGGATAACAATTTCACACAGGAAACAGCTATGACCATGATT
ACGCCAAGCTTGCATGCAAATTCTATTTCAAGGAGACAGTCATAATGAAATACCTATTGCCTACGGCAGCCGCTGG
ATTGTTATTACTCGCGGCCCAGCCGGCCATGGCTCTAGAGTAAGGAGGCAGTCATAATGAAGTACCTTTTGCCAAC
GGCTGCCGCTGGCTTGTTATTGCTCGCGGCACAGCCGGCAATTGGGCGCGCCTAGTCGACCAAGGGCCCATCGGTC
TTCCCCCTGGCACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAAAGTTGAGCCCAAATCTTGTGACAAAGCGGCCGCTAGCA
CTGTTGAAAGTTGTTTAGCAAAACCTCATACAGAAAATTCATTTACTAACGTCTGGAAAGACGACAAAACTTTAGA
TCGTTACGCTAACTATGAGGGCTGTCTGTGGAATGCTACGGGCGTTGTGGTTTGCACTGGTGACGAAACTCAGTGT
TACGGTACATGGGTTCCTATTGGGCTTGCTATCCCTGAAAATGAGGGTGGTGGCTCTGAGGGTGGCGGTTCTGAGG
GTGGCGGTTCTGAGGGTGGCGGTACTAAACCTCCAGAGTACGGTGATACACCTATTCCGGGCTATACTTATATCAA
CCCTCTCGACGGCACTTATCCGCCTGGTACTGAGCAAAACCCCGCTAATCCTAATCCTTCTCTTGAGGAGTCTCAG
CCTCTTAATACTTTCATGTTTCAGAATAATAGGTTCCGAAATAGGCAGGGTGCATTAACTGTTTATACGGGCACTG
TTACTCAAGGCACTGACCCCGTTAAAACTTATTACCAGTACACTCCTGTATCATCAAAAGCCATGTATGACGCTTA
CTGGAACGGTAAATTCAGAGACTGCGCTTTCCATTCTGGCTTTAATGAGGATCCATTCGTTTGTGAATATCAAGGC
CAATCGTCTGACCTGCCTCAACCTCCTGTCAATGCTGGCGGCGGCTCTGGTGGTGGTTCTGGTGGCGGCTCTGAGG
GTGGCGGCTCTGAGGGTGGCGGTTCTGAGGGTGGCGGCTCTGAGGGTGGCGGTTCCGGTGGCGGCTCCGGTTCCGG
TGATTTTGATTATGAAAAAATGGCAAACGCTAATAAGGGGGCTATGACCGAAAATGCCGATGAAAACGCGCTACAG
TCTGACGCTAAAGGCAAACTTGATTCTGTCGCTACTGATTACGGTGCTGCTATCGATGGTTTCATTGGTGACGTTT
CCGGCCTTGCTAATGGTAATGGTGCTACTGGTGATTTTGCTGGCTCTAATTCCCAAATGGCTCAAGTCGGTGACGG
TGATAATTCACCTTTAATGAATAATTTCCGTCAATATTTACCTTCTTTGCCTCAGTCGGTTGAATGTCGCCCTTAT
GTCTTTGGCGCTGGTAAACCATATGAATTTTCTATTGATTGTGACAAAATAAACTTATTCCGTGGTGTCTTTGCGT
TTCTTTTATATGTTGCCACCTTTATGTATGTATTTTCGACGTTTGCTAACATACTGCGTAATAAGGAGTCTTAATA
AGCTAGCCATCACCACCATCATCACTAATAATGAAAGCCCGCCTAATGAGCGGGCTTTTTTTTGAATTCACTGGCC
GTCGTTTTACAACGTCGTGACTGGGAAAACCCTGGCGTTACCCAACTTAATCGCCTTGCAGCACATCCCCCTTTCG
CCAGCTGGCGTAATAGCGAAGAGGCCCGCACCGATCGCCCTTCCCAACAGTTGCGCAGCCTGAATGGCGAATGGCG
CCTGATGCGGTATTTTCTCCTTACGCATCTGTGCGGTATTTCACACCGCATACGTCAAAGCAACCATAGTACGCGC
CCTGTAGCGGCGCATTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTGATTTGGGTGATGGTTCACGTA
GTGGGCCATCGCCCTGATAGACGGTTTTTCGCCCTTTGACGTTGGAGTCCACGTTCTTTAATAGTGGACTCTTGTT
CCAAACTGGAACAACACTCAACCCTATCTCGGGCTATTCTTTTGATTTATAAGGGATTTTGCCGATTTCGGCCTAT
TGGTTAAAAAATGAGCTGATTTAACAAAAATTTAACGCGAATTTTAACAAAATATTAACGTTTACAATTTTATGGT
GCACTCTCAGTACAATCTGCTCTGATGCCGCATAGTTAAGCCAGCCCCGACACCCGCCAACACCCGCTGACGCGCC
CTGACGGGCTTGTCTGCTCCCGGCATCCGCTTACAGACAAGCTGTGACCGTCTCCGGGAGCTGCATGTGTCAGAGG
TTTTCACCGTCATCACCGAAACGCGCGAGACGAAAGGGCCTCGTGATACGCCTATTTTTATAGGTTAATGTCATGA
TAATAATGGTTTCTTAGACGTCAGGTGGCACTTTTCGGGGAAATGTGCGCGGAACCCCTATTTGTTTATTTTTCTA
AATACATTCAAATATGTATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATCGAACTGGATCTCAACAG
CGGTAAGATCCTTGAGAGTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTCTCAGAATGACTTGGTTG
AGTACTCACCAGTCACAGAAAAGCATCTTACGGATGGCATGACAGTAAGAGAATTATGCAGTGCTGCCATAACCAT
GAGTGATAACACTGCGGCCAACTTACTTCTGACAACGATCGGAGGACCGAAGGAGCTAACCGCTTTTTTGCACAAC
ATGGGGGATCATGTAACTCGCCTTGATCGTTGGGAACCGGAGCTGAATGAAGCCATACCAAACGACGAGCGTGACA
CCACGATGCCTGCAGCAATGGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTAAGCCCT
CCCGTATCGTAGTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATGAGAAAGCGCCACGCTTC
CCGAAGGGAGAAAGGCGGACAGGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAGCGTCGATTTTTGTGATGCTCG
TCAGGGGGGCGGAGCCTATGGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTTTGAGTGAGCTGATACCGC
TCGCCGCAGCCGAACGACCGAGCGCAGCGAGTCAGTGAGCGAGGAAGC

1.5.11:
Name of vector: pMAB103
Alteration from pMAB87: Insertion of anti-TT specific Fab fragment.

Same as for [SEQ. ID. NO. 18], except that bases 319-754 were replaced with the following sequence [SEQ. ID. NO. 19]:

GGCCCAGCCGGCCATGGCTGACATCCAGATGACCCAGTCTCCATCCTCCCTGTCTGCATCTGTAGGAGACAGAGTC
ATCATCACTTGCCGGGCAAGTCAGAGTATTAGCACCTATTTAAATTGGTATCAGCAGAAACCAGGGAAAGCCCCTA
AACTCCTGATCTATTATGCAACCAATTTGCAAAGTGGGGTCCCATCAAGGTTCAGTGGCAGTGGATCTGGGACAGA
TTTCACTCTCACCATCAGCAGTCTGCAACCTGAAGATTTTGCGACTTATTATTGTCAACAGAGTTCCAACACCGTC
ACTTTCGGCCCTGGGACCAAAGTGGATATGAAGCGAACTGTGGCTGCACCATCTGTCTTCATCTTCCCGCCATCTG
ATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCC
ATCAGGGCCTGAGTTCGCCCGTCACAAAGAGCTTCAACAGGGGAGAGTGTTAATTCTAGAGTAAGGAGGCAGTCAT
AATGAAGTACCTTTTGCCAACGGCTGCCGCTGGCTTGTTATTGCTCGCGGCACAGCCGGCAATTGCCCAGGTGCAG
CTGGTGCAGTCTGGGGGAGGCTTGGTACAGCCTGGGGGGTCCGTGAGACTCTCCTGTGCAGCCTCTGGATTCAGTT
TTAGCAGCTATGCCATGAGCTGGGTCCGCCAGGCTCCAGGGATGGGGCTGGAGTGGGTCGCGGCTATTAGTGCTAG
AGGAACTACCACATATTATGCAGACTCCGTGACGGGCCGATTGACCATCTCCAGAGACAATTCCATGAACACGCTA
TATCTGCACTTGAACAGCCTGAGAGCCGAGGACACGGCCGTTTATTACTGTGCGAAAGCGGGAAAACAGTGGCTGG
CCCACTACTACTTTGACTCCTGGGGCCAGGGAACCCTGGTCACCGTCTCCTCAGCCTCCACCAAGGGCCCATCGGT
CTTCCCCCTGGCACCCTCCTCCAAGAGCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAA
CGTGAATCACAAGCCCAGCAACACCAAGGTGGACAAGAAAGTTGAGCCCAAATCTTGTGACAAAGCGGCCGC

1.6 Using Phaberge to Isolate TT-Specific Clones from a Donor-Derived Library

A phagemid library was constructed from blood donor material and used in combination with helper phage Phaberge to isolate TT-specific clones by biopanning:

Three volunteers each donated 50 mL of blood, from which human peripheral blood leukocytes (PBL's) were isolated and frozen down. From each thawed sample, poly-A RNA was isolated (Ambion's “Poly(A) Pure mRNA purification Kit”) and used in RT-PCR (“Thermoscript RT-PCR System”; Invitrogen-GibcoBRL) to amplify immunoglobulin gene fragments: Both V_{H}-fragments (families V_{H}1, V_{H}3, V_{H}4, V_{H}5, and V_{H}6) and diverse V_{L}C_{L} fragments (different VκCκ and VλCλ families) were amplified. These fragments were cloned into vector pMAB87 in a consecutive fashion, first V_{L}C_{L}-fragments and then V_{H}-fragments. Following electroporation into E. coli strain TOP10F′, the resulting final library had approximately 10⁸ different clones.

From this library, we prepared phagemid virions as described in Section A.1.3 using helper phage Phaberge. These phagemid virions were used in biopanning to enrich for TT-specific clones. Binding conditions for biopanning were essentially the same as those for ELISA (section A.1.4.1), but binding buffer consisted of: 50 mM tris, 150 mM NaCl, 1mM MgCl₂, 1 mM CaCl₂, 0.2% Tween-20, 1% BSA, 0.03% NaN₃, pH7.4. The number of virions per microtiter well varied from 8×10⁹ (in the first round of biopanning) to 2×10⁵ (in the final round). After the binding step and multiple washes, elution was done in two steps, the first using 76 mM sodium citrate pH2.4 for 30 minutes and the second using 50 mM HCl for 30-45 seconds, then followed by pH neutralization of pooled eluates by 2 M tris pH8.0. Eluted phagemid virions were propagated in TOP10F′ cells. A total of four rounds of biopanning were performed. Isolated individual phagemid clones were tested for specific binding to TT by whole phage ELISA, as described in Section A.1.4.1, and the corresponding phagemid DNA was prepared from 5 mL cultures (Qiagen miniprep kit) and subjected to DNA sequencing.

B. Results

1.1 Effect of Different Parameters on Phage Production and Display

In a first set of experiments (FIG. 1), we varied a number of basal conditions to obtain high production of phagemid virion and efficient viral display of a TT-specific Fab fragment. This optimization was done first, followed by further improvements (Sections B.1.2 to B.1.5) by a genetically modifying a helper phage.

In the first round of optimization, five different parameters were altered, one at a time: Different phagemid constructs, bacterial host strains, different commercially available helper phage, media additions and growth temperatures. Each altered parameter was compared with a fixed standard condition: see Table II for details.

First, different phagemid vectors were compared: pMAB29, pMAB77 and pMAB66. These vectors encode for the same anti-TT Fab, but differ in other features: see FIG. 2B and Sections A.1.5.1-1.5.11. The first vector, pMAB29, does not contain a rho-dependent transcriptional terminator 3′ of the bi-cistronic Fab-gene 3 operon. In attempts to increase Fab-g3p expression, such a terminator was inserted in both the vectors pMAB77 and pMAB66. Also, to ascertain if the length of gene 3 has an effect on phage production, it was either truncated at the 5′-end (pMAB66) or remained as full length (pMAB77), i.e.: Of the 406 amino acids present in the natural leader-less g3p, amino acids 211-406 are present in pMAB66, and amino acids 3-406 are present in pMAB77. Finally, both pMAB77 and pMAB66 have identical, minor changes as compared to pMAB29: they have two NheI sites that flank gene 3 as well as an un-translated poly-histidine sequence (“His₆”) immediately 3′ of gene 3. These features enable production of soluble poly-histidine-tagged Fab fragments after removal of gene 3 by NheI-digestion (data not shown); Also, the c-myc tag present in pMAB29 is not present in vectors pMAB66 and pMAB77.

After infection with R408 helper phage, the three phagemids (pMAB29, pMAB77 and pMAB66) gave comparable number of phagemid virion, approximately 10¹⁰/mL, as tested in a CFU assay (Table II A). Display of Fab-g3p was tested by ELISA and showed that vectors pMAB66 and pMAB77 were both better than pMAB29 (Table II A). The length of gene 3 did not appear to have a major impact, since the relatively small difference in display between pMAB66 and pMAB77 was within the variation seen in repeat experiments.

In a final attempt to improve functionality of phagemids, we substituted the plasmid origin of replication of pMAB77 (pUC/ColE1) for a low-copy moiety, pBR322. This resulted in the novel phagemid pMAB103. Previous data [36] suggest that a low-copy phagemid poses less of a burden to the bacterial host than does a high-copy phagemid, and also, does not compromise viral display levels; these two factors enabled more efficient selection of desired clones from a library. In accordance with these data, we found (Table II A) that pMAB103 had similar display level to pMAB77. Also, pMAB103 produced a larger number of phagemid virions than did pMAB77, which is an unexpected improvement. A possible explanation for increased virion production is a difference in the ratio of vector copy number: since pMAB103 is a “low-copy vector”, it is likely that the ratio of helper phage genomes to phagemids is relatively high, and that the corresponding ration for pMAB77 might be lower. Therefore, in the case of pMAB103 there would be relatively more helper phage gene products to assemble phagemid virions then there would be in the case of pMAB77.

Next, the effects of media additions and helper phage were tested (Table II: A and B). Addition of 1 mM IPTG (isopropyl β-D-thio-galacto-pyranoside) did not increase CFU titers or display levels. In fact, it had a negative effect on display by phagemid pMAB66. When substituting the helper phage, both M13K07 and VCS-M13 gave comparable production of Amp-resistant phage as R408 (Table II B). Display levels were comparable for R408 and VCS-M13. For M13K07, they were slightly higher but still not reproducibly so. Both M13K07 and VCS-M13 confer resistance to kanamycin. However, including this antibiotic after the addition of either M13K07 or VCS-M13 had either no effects on phage production or display or had negative effects (data not shown).

Four different bacterial host strains were compared (Table II: C). Production of phage was similar with XL-1 Blue MRF′, SURE and TOP10F′, but TG-1 was clearly inferior. Display levels varied between hosts, with TOP10F′ being the best.

When growing bacteria at different temperatures after helper phage infection (Table II: D), we again found no substantial effect on CFU titers. However, the display level was higher at 30-32° C. then at 37° C.

1.2 Mutated Helper Phage (“Phaberge”): Verification of Identity

To further improve phage display technology (see section “summary of the Invention”), the helper phage M13K07 was mutagenized. First, the helper phage created by gene cloning were tested to see if they indeed contained the correct mutation (see Materials & Methods). After ligation and transformation, plaques were selected and screened by a combination of bacterial PCR and analytical digestion with restriction enzyme DdeI. The resulting DNA fragments had sizes distinctly different from those of M13KO7 and compatible with a construct containing the desired mutation (data not shown).

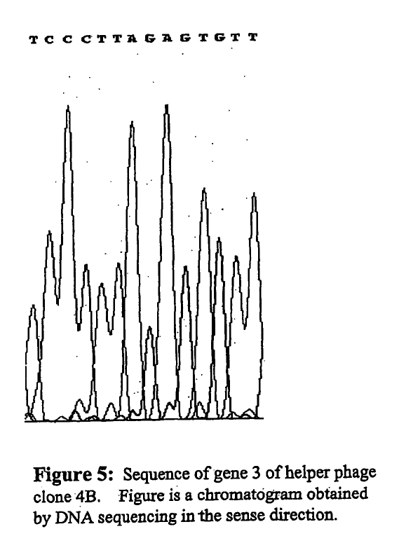

To ensure purity of novel helper phage constructs, a new PFU assay was performed, using a suspension of one plaque to infect indicator bacteria: A new, well isolated plaque (“clone 4B”) was picked and grown in liquid media. From this new culture, we isolated both double-stranded helper phage DNA (from bacteria) and phage particles (from culture supernatant). Sequencing of DNA confirmed that the desired mutation was indeed present (FIG. 5—same sequence as in FIG. 1B). Also, the DNA from clone 4B was digested by restriction enzymes ClaI and HaelI and found to have the same gross structure as M13K07, as expected if the mutation was discrete (data not shown). Altogether, these data show that clone 4B contains the desired mutation, and that it does not have any other obvious difference from M13KO7. The fact that the gross structure of clone 4B was not different from M13K07, despite having undergone two consecutive rounds of PFU assay and propagation in liquid culture, suggests that the genome of clone 4B is relatively stable.

Also, by using a CFU assay with kanamycin-containing agar plates, we found that clone 4B conferred kanamycin-resistance, as did M13K07.

Finally, the entire sequence of clone 4B was established. The sequence of the elements that make up M13K07 (and therefore also clone 4B) are known from the prior art, i.e.: the M13 genome, the kanamycin resistance (kan^{R}) gene and the p15^{A} origin of replication (FIG. 1A). However, the junctions between these three elements have only been schematically described: (reference 23). To establish the exact sequence of clone 4B DNA sequencing was performed across these three junctions. By combining the sequencing results with those in the prior art, the full sequence of clone 4B was assembled:

| | |
|---|---|
| GTGAAAAAATTATTATTCGCAATTCCTTTAGTTGTTCCTTTCTATTCTCACTCCGCTGAAAC | (SEQ. ID. NO 20) |
| TGTTGAAAGTTGTTTAGCAAAACCCCATACAGAAAATTCATTTACTAACGTCTGGAAAGACG | |
| ACAAAACTTTAGATCGTTACGCTAACTATGAGGGTTGTCTGTGGAATGCTACAGGCGTTGTA | |
| GTTTGTACTGGTGACGAAACTCAGTGTTACGGTACATGGGTTCCTATTGGGCTTGCTATCCC | |
| TGAAAATGAGGGTGGTGGCTCTGAGGGTGGCGGTTCTGAGGGTGGCGGTTCTGAGGGTGGCG | |
| GTACTAAACCTCCTGAGTACGGTGATACACCTATTCCGGGCTATACTTATATCAACCCTCTC | |
| GACGGCACTTATCCGCCTGGTACTGAGCAAAACCCCGCTAATCCTAATCCTTCTCTTGAGGA | |
| GTCTCAGCCTCTTAATACTTTCATGTTTCAGAATAATAGGTTCCGAAATAGGCAGGGGGCAT | |
| TAACTGTTTATACGGGCACTGTTACTCAAGGCACTGACCCCGTTAAAACTTATTACCAGTAC | |
| ACTCCTGTATCATCAAAAGCCATGTATGACGCTTACTGGAACGGTAAATTCAGAGACTGCGC | |
| TTTCCATTCTGGCTTTAATGAGGATCCATTCGTTTGTGAATATCAAGGCCAATCGTCTGACC | |
| TGCCTCAACCTCCTGTCAATGCTGGCGGCGGCTCTGGTGGTGGTTCTGGTGGCGGCTCTGAG | |
| GGTGGTGGCTCTGAGGGTGGCGGTTCTGAGGGTGGCGGCTCTGAGGGAGGCGGTTCCGGTGG | |
| TGGCTCTGGTTCCGGTGATTTTGATTATGAAAAGATGGCAAACGCTAATAAGGGGGCTATGA | |
| CCGAAAATGCCGATGAAAACGCGCTACAGTCTGACGCTAAAGGCAAACTTGATTCTGTCGCT | |
| ACTGATTACGGTGCTGCTATCGATGGTTTCATTGGTGACGTTTCCGGCCTTGCTAATGGTAA | |
| TGGTGCTACTGGTGATTTTGCTGGCTCTAATTCCCAAATGGCTCAAGTCGGTGACGGTGATA | |
| ATTCACCTTTAATGAATAATTTCCGTCAATATTTACCTTCCCTCCCTTAGAGTGTTGAATGT | |
| CGCCCTTTTGTCTTTGGCGCTGGTAAACCATATGAATTTTCTATTGATTGTGACAAAATAAA | |
| CTTATTCCGTGGTGTCTTTGCGTTTCTTTTATATGTTGCCACCTTTATGTATGTATTTTCTA | |
| CGTTTGCTAACATACTGCGTAATAAGGAGTCTTAATCATGCCAGTTCTTTTGGGTATTCCGT | |
| TATTATTGCGTTTCCTCGGTTTCCTTCTGGTAACTTTGTTCGGCTATCTGCTTACTTTTCTT | |
| AAAAAGGGCTTCGGTAAGATAGCTATTGCTATTTCATTGTTTCTTGCTCTTATTATTGGGCT | |
| TAACTCAATTCTTGTGGGTTATCTCTCTGATATTAGCGCTCAATTACCCTCTGACTTTGTTC | |
| AGGGTGTTCAGTTAATTCTCCCGTCTAATGCGCTTCCCTGTTTTTATGTTATTCTCTCTGTA | |
| AAGGCTGCTATTTTCATTTTTGACGTTAAACAAAAAATCGTTTCTTATTTGGATTGGGATAA | |
| ATAATATGGCTGTTTATTTTGTAACTGGCAAATTAGGCTCTGGAAAGACGCTCGTTAGCGTT | |
| GGTAAGATTCAGGATAAAATTGTAGCTGGGTGCAAAATAGCAACTAATCTTGATTTAAGGCT | |
| TCAAAACCTCCCGCAAGTCGGGAGGTTCGCTAAAACGCCTCGCGTTCTTAGAATACCGGATA | |
| AGCCTTCTATATCTGATTTGCTTGCTATTGGGCGCGGTAATGATTCCTACGATGAAAATAAA | |
| AACGGCTTGCTTGTTCTCGATGAGTGCGGTACTTGGTTTAATACCCGTTCTTGGAATGATAA | |
| GGAAAGACAGCCGATTATTGATTGGTTTCTACATGCTCGTAAATTAGGATGGGATATTATTT | |
| TTCTTGTTCAGGACTTATCTATTGTTGATAAACAGGCGCGTTCTGCATTAGCTGAACATGTT | |
| GTTTATTGTCGTCGTCTGGACAGAATTACTTTACCTTTTGTCGGTACTTTATATTCTCTTAT | |
| TACTGGCTCGAAAATGCCTCTGCCTAAATTACATGTTGGCGTTGTTAAATATGGCGATTCTC | |
| AATTAAGCCCTACTGTTGAGCGTTGGCTTTATACTGGTAAGAATTTGTATAACGCATATGAT | |
| ACTAAACAGGCTTTTTCTAGTAATTATGATTCCGGTGTTTATTCTTATTTAACGCCTTATTT | |
| ATCACACGGTCGGTATTTCAAACCATTAAATTTAGGTCAGAAGATGAAATTAACTAAAATAT | |
| ATTTGAAAAAGTTTTCTCGCGTTCTTTGTCTTGCGATTGGATTTGCATCAGCATTTACATAT | |
| AGTTATATAACCCAACCTAAGCCGGAGGTTAAAAAGGTAGTCTCTCAGACCTATGATTTTGA | |
| TAAATTCACTATTGACTCTTCTCAGCGTCTTAATCTAAGCTATCGCTATGTTTTCAAGGATT | |
| CTAAGGGAAAATTAATTAATAGCGACGATTTACAGAAGCAAGGTTATTCACTCACATATATT | |
| GATTTATGTACTGTTTCCATTAAAAAAGGTAATTCAAATGAAATTGTTAAATGTAATTAATT | |
| TTGTTTTCTTGATGTTTGTTTCATCATCTTCTTTTGCTCAGGTAATTGAAATGAATAATTCG | |
| CCTCTGCGCGATTTTGTAACTTGGTATTCAAAGCAATCAGGCGAATCCGTTATTGTTTCTCC | |
| CGATGTAAAAGGTACTGTTACTGTATATTCATCTGACGTTAAACCTGAAAATCTACGCAATT | |
| TCTTTATTTCTGTTTTACGTGCTAATAATTTTGATATGGTTGGTTCAATTCCTTCCATAATT | |
| CAGAAGTATAATCCAAACAATCAGGATTATATTGATGAATTGCCATCATCTGATAATCAGGA | |
| ATATGATGATAATTCCGCTCCTTCTGGTGGTTTCTTTGTTCCGCAAAATGATAATGTTACTC | |
| AAACTTTTAAAATTAATAACGTTCGGGCAAAGGATTTAATACGAGTTGTCGAATTGTTTGTA | |
| AAGTCTAATACTTCTAAATCCTCAAATGTATTATCTATTGACGGCTCTAATCTATTAGTTGT | |
| TAGTGCACCTAAAGATATTTTAGATAACCTTCCTCAATTCCTTTCTACTGTTGATTTGCCAA | |
| CTGACCAGATATTGATTGAGGGTTTGATATTTGAGGTTCAGCAAGGTGATGCTTTAGATTTT | |
| TCATTTGCTGCTGGCTCTCAGCGTGGCACTGTTGCAGGCGGTGTTAATACTGACCGCCTCAC | |
| CTCTGTTTTATCTTCTGCTGGTGGTTCGTTCGGTATTTTTAATGGCGATGTTTTAGGGCTAT | |
| CAGTTCGCGCATTAAAGACTAATAGCCATTCAAAAATATTGTCTGTGCCACGTATTCTTACG | |
| CTTTCAGGTCAGAAGGGTTCTATCTCTGTTGGCCAGAATGTCCCTTTTATTACTGGTCGTGT | |
| GACTGGTGAATCTGCCAATGTAAATAATCCATTTCAGACGATTGAGCGTCAAAATGTAGGTA | |
| TTTCCATGAGCGTTTTTCCTGTTGCAATGGCTGGCGGTAATATTGTTCTGGATATTACCAGC | |
| AAGGCCGATAGTTTGAGTTCTTCTACTCAGGCAAGTGATGTTATTACTAATCAAAGAAGTAT | |
| TGCTACAACGGTTAATTTGCGTGATGGACAGACTCTTTTACTCGGTGGCCTCACTGATTATA | |
| AAAACACTTCTCAAGATTCTGGCGTACCGTTCCTGTCTAAAATCCCTTTAATCGGCCTCCTG | |
| TTTAGCTCCCGCTCTGATTCCAACGAGGAAAGCACGTTATACGTGCTCGTCAAAGCAACCAT | |
| AGTACGCGCCCTGTAGCGGCGCATTAAGCGCGGCGGGTGTGGTGGTTACGCGCAGCGTGACC | |
| GCTACACTTGCCAGCGCCCTAGCGCCCGCTCCTTTCGCTTTCTTCCCTTCCTTTCTCGCCAC | |
| GTTCGCCGGCTTTCCCCGTCAAGCTCTAAATCGGGGGCTCCCTTTAGGGTTCCGATTTAGTG | |
| CTTTACGGCACCTCGACCCCAAAAAACTTGATTTGGGTGATGGTTCACGTAGTGGGCCATCG | |
| CCCTGATAGACGGTTTTTCGCCCTTTGACGTTGGAGTCCACGTTCTTTAATAGTGGACTCTT | |
| GTTCCAAACTGGAACAACACTCAACCCTATCTCGGGACGGATCGCTTCATGTGGCAGGAGAA | |
| AAAAGGCTGCACCGGTGCGTCAGCAGAATATGTGATACAGGATATATTCCGCTTCCTCGCTC | |
| ACTGACTCGCTACGCTCGGTCGTTCGACTGCGGCGAGCGGAAATGGCTTACGAACGGGGCGG | |
| AGATTTCCTGGAAGATGCCAGGAAGATACTTAACAGGGAAGTGAGAGGGCCGCGGCAAAGCC | |
| GTTTTTCCATAGGCTCCGCCCCCCTGACAAGCATCACGAAATCTGACGCTCAAATCAGTGGT | |
| GGCGAAACCCGACAGGACTATAAAGATACCAGGCGTTTCCCCCTGGCGGCTCCCTCGTGCGC | |
| TCTCCTGTTCCTGCCTTTCGGTTTACCGGTGTCATTCCGCTGTTATGGCCGCGTTTGTCTCA | |
| TTCCACGCCTGACACTCAGTTCCGGGTAGGCAGTTCGCTCCAAGCTGGACTGTATGCACGAA | |
| CCCCCCGTTCAGTCCGACCGCTGCGCCTTATCCGGTAACTATCGTCTTGAGTCCAACCCGGA | |
| AAGACATGCAAAAGCACCACTGGCAGCAGCCACTGGTAATTGATTTAGAGGAGTTAGTCTTG | |
| AAGTCATGCGCCGGTTAAGGCTAAACTGAAAGGACAAGTTTTGGTGACTGCGCTCCTCCAAG | |
| CCAGTTACCTCGGTTCAAAGAGTTGGTAGCTCAGAGAACCTTCGAAAAACCGCCCTGCAAGG | |
| CGGTTTTTTCGTTTTCAGAGCAAGAGATTACGCGCAGACCAAAACGATCTCAAGAAGATCAT | |
| CTTATTAAGGGGTCTGACGCTCAGTGGAACGAAAACTCACGTTAAGGGATTTTGGTCATGAG | |
| ATTATCAAAAGGATCTTCACCTAGATCCTTTTAAATTAAAAATGAAGTTTTTAAATCAATCT | |
| AAAGTATATATGAGTAAACTTGGTCTGACAGTTACCAATGCTTAATCAGTGAGGCACCTATC | |
| TCAGCGATCTGTCTATTTCGTTCATCCATAGTTGCCTGACTCCCCGTCGTGTAGATAACTAC | |
| GATACGGGAGGGCTTACCATCTGGCCCCAGTGCTGCAATGATACCGCGAGACCCACGCTCAC | |
| CGGCTCCAGATTTATCAGCAATAAACCAGCCAGCCGATTCGAGCTCGCCCGGGGATCGACCA | |
| GTTGGTGATTTTGAACTTTTGCTTTGCCACGGAACGGTCTGCGTTGTCGGGAAGATGCGTGA | |
| TCTGATCCTTCAACTCAGCAAAAGTTCGATTTATTCAACAAAGCCGCCGTCCCGTCAAGTCA | |
| GC | |
| GTAATGCTCTGCCAGTGTTACAACCAATTAACCAATTCTGATTAGAAAAACTCATCGAGCAT | |
| CAAATGAAACTGCAATTTATTCATATCAGGATTATCAATACCATATTTTTGAAAAAGCCGTT | |
| TCTGTAATGAAGGAGAAAACTCACCGAGGCAGTTCCATAGGATGGCAAGATCCTGGTATCGG | |
| TCTGCGATTCCGACTCGTCCAACATCAATACAACCTATTAATTTCCCCTCGTCAAAAATAAG | |
| GTTATCAAGTGAGAAATCACCATGAGTGACGACTGAATCCGGTGAGAATGGCAAAAGCTTAT | |
| GCATTTCTTTCCAGACTTGTTCAACAGGCCAGCCATTACGCTCGTCATCAAAATCACTCGCA | |
| TCAACCAAACCGTTATTCATTCGTGATTGCGCCTGAGCGAGACGAAATACGCGATCGCTGTT | |
| AAAAGGACAATTACAAACAGGAATCGAATGCAACCGGCGCAGGAACACTGCCAGCGCATCAA | |
| CAATATTTTCACCTGAATCAGGATATTCTTCTAATACCTGGAATGCTGTTTTCCCGGGGATC | |
| GCAGTGGTGAGTAACCATGCATCATCAGGAGTACGGATAAAATGCTTGATGGTCGGAAGAGG | |
| CATAAATTCCGTCAGCCAGTTTAGTCTGACCATCTCATCTGTAACATCATTGGCAACGCTAC | |
| CTTTGCCATGTTTCAGAAACAACTCTGGCGCATCGGGCTTCCCATACAATCGATAGATTGTC | |
| GCACCTGATTGCCCGACATTATCGCGAGCCCATTTATACCCATATAAATCAGCATCCATGTT | |
| GGAATTTAATCGCGGCCTCGAGCAAGACGTTTCCCGTTGAATATGGCTCATAACACCCCTTG | |
| TATTACTGTTTATGTAAGCAGACAGTTTTATTGTTCATGATGATATATTTTTATCTTGTGCA | |
| ATGTAACATCAGAGATTTTGAGACACAACGTGGCTTTCCCCCCCCCCCCCCTGAAGGTGTGG | |
| GCCTATTCTTTTGATTTATAAGGGATTTTGCCGATTTCGGCCTATTGGTTAAAAAATGAGCT | |
| GATTTAACAAAAATTTAACGCGAATTTTAACAAAATATTAACGTTTACAATTTAAATATTTG | |
| CTTATACAATCTTCCTGTTTTTGGGGCTTTTCTGATTATCAACCGGGGTACATATGATTGAC | |
| ATGCTAGTTTTACGATTACCGTTCATCGATTCTCTTGTTTGCTCCAGACTCTCAGGCAATGA | |
| CCTGATAGCCTTTGTAGACCTCTCAAAAATAGCTACCCTCTCCGGCATGAATTTATCAGCTA | |
| GAACGGTTGAATATCATATTGATGGTGATTTGACTGTCTCCGGCCTTTCTCACCCTTTTGAA | |
| TCTTTACCTACACATTACTCAGGCATTGCATTTAAAATATATGAGGGTTCTAAAAATTTTTA | |
| TCCTTGCGTTGAAATAAAGGCTTCTCCCGCAAAAGTATTACAGGGTCATAATGTTTTTGGTA | |
| CAACCGATTTAGCTTTATGCTCTGAGGCTTTATTGCTTAATTTTGCTAATTCTTTGCCTTGC | |
| CTGTATGATTTATTGGATGTTAACGCTACTACTATTAGTAGAATTGATGCCACCTTTTCAGC | |
| TCGCGCCCCAAATGAAAATATAGCTAAACAGGTTATTGACCATTTGCGAAATGTATCTAATG | |
| GTCAAACTAAATCTACTCGTTCGCAGAATTGGGAATCAACTGTTACATGGAATGAAACTTCC | |
| AGACACCGTACTTTAGTTGCATATTTAAAACATGTTGAGCTACAGCACCAGATTCAGCAATT | |
| AAGCTCTAAGCCATCCGCAAAAATGACCTCTTATCAAAAGGAGCAATTAAAGGTACTCTCTA | |
| ATCCTGACCTGTTGGAGTTTGCTTCCGGTCTGGTTCGCTTTGAAGCTCGAATTAAAACGCGA | |
| TATTTGAAGTCTTTCGGGCTTCCTCTTAATCTTTTTGATGCAATCCGCTTTGCTTCTGACTA | |
| TAATAGTCAGGGTAAA | |
| GACCTGATTTTTGATTTATGGTCATTCTCGTTTTCTGAACTGTTTAAAGCATTTGAGGGGGA | |
| TTCAATGAATATTTATGACGATTCCGCAGTATTGGACGCTATCCAGTCTAAACATTTTACTA | |
| TTACCCCCTCTGGCAAAACTTCTTTTGCAAAAGCCTCTCGCTATTTTGGTTTTTATCGTCGT | |
| CTGGTAAACGAGGGTTATGATAGTGTTGCTCTTACTATGCCTCGTAATTCCTTTTGGCGTTA | |
| TGTATCTGCATTAGTTGAATGTGGTATTCCTAAATCTCAACTGATGAATCTTTCTACCTGTA | |
| ATAATGTTGTTCCGTTAGTTCGTTTTATTAACGTAGATTTTTCTTCCCAACGTCCTGACTGG | |
| TATAATGAGCCAGTTCTTAAAATCGCATAAGGTAATTCACAATGATTAAAGTTGAAATTAAA | |
| CCATCTCAAGCCCAATTTACTACTCGTTCTGGTGTTTCTCGTCAGGGCAAGCCTTATTCACT | |
| GAATGAGCAGCTTTGTTACGTTGATTTGGGTAATGAATATCCGGTTCTTGTCAAGATTACTC | |
| TTGATGAAGGTCAGCCAGCCTATGCGCCTGGTCTGTACACCGTTCATCTGTCCTCTTTCAAA | |
| GTTGGTCAGTTCGGTTCCCTTATGATTGACCGTCTGCGCCTCGTTCCGGCTAAGTAACATGG | |
| AGCAGGTCGCGGATTTCGACACAATTTATCAGGCGATGATACAAATCTCCGTTGTACTTTGT | |
| TTCGCGCTTGGTATAATCGCTGGGGGTCAAAGATGAGTGTTTTAGTGTATTCTTTCGCCTCT | |
| TTCGTTTTAGGTTGGTGCCTTCGTAGTGGCATTACGTATTTTACCCGTTTAATGGAAACTTC | |
| CTCATGAAAAAGTCTTTAGTCCTCAAAGCCTCTGTAGCCGTTGCTACCCTCGTTCCGATGCT | |
| GTCTTTCGCTGCTGAGGGTGACGATCCCGCAAAAGCGGCCTTTAACTCCCTGCAAGCCTCAG | |
| CGACCGAATATATCGGTTATGCGTGGGCGATGGTTGTTGTCATTGTCGGCGCAACTATCGGT | |
| ATCAAGCTGTTTAAGAAATTCACCTCGAAAGCAAGCTGATAAACCGATACAATTAAAGGCTC | |
| CTTTTGGAGCCTTTTTTTTTGGAGATTTTCAAC | |

1.3 Production and Replication of Phaberge

The mutant helper phage was able to replicate, since it produced plaques in repeated PFU assays (Section B.1.2). Additional PFU assays were performed with clone 4B, a.k.a. Phaberge, to test how much helper phage virions was produced, and if it, as expected, could only propagate in SupE⁺ bacterial hosts (e.g. XL-1 Blue MRF′).

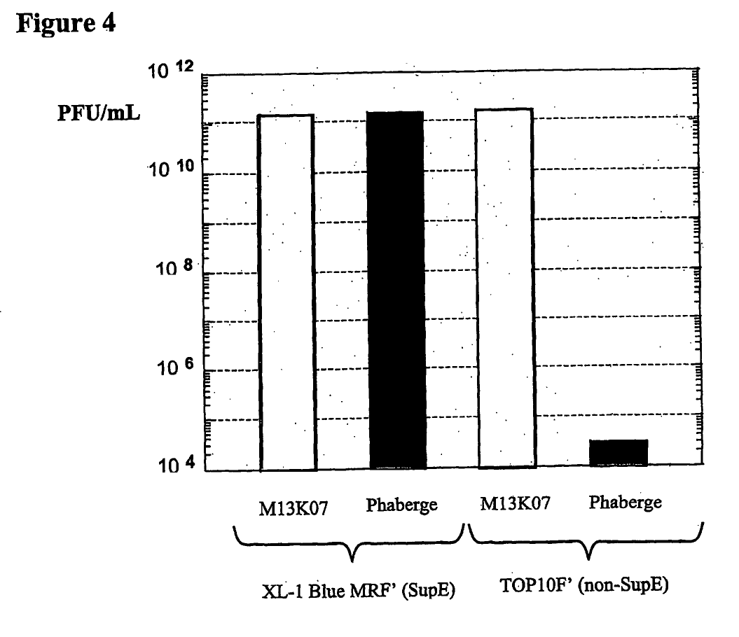

As shown in FIG. 4, Phaberge was produced at similar level as its non-mutated predecessor, M13K07. Repeat experiments were somewhat variable, but the PFU-titer of Phaberge was typically within an order of magnitude of that of M13K07. Importantly, Phaberge showed efficient replication only in a SupE⁺ bacterial host, but, as a control, M13K07 replicated equally well in SupE⁺ and non-SupE hosts.

Thus, Phaberge is produced at high levels, replicates well and its replication is restricted to a SupE⁺ host.

1.4 Helper Phage Function of Phaberge

Next, we tested if Phaberge indeed had helper phage function, i.e.: if it could supplement phagemid-containing bacteria in producing phage particles containing phagemid vector (“phagemid virion”). To test for various aspects of helper phage function TOP10F′ bacteria (non-SupE) housing different phagemid vectors was used: see below. Using similar methods as in Section B.1.1, it was tested how much phagemid virion was produced by supplementing these phagemids with either helper phage Phaberge or M13K07. These experiments are exemplified in Table III. In Table III, experiments 1 and 2A it was found that Phaberge indeed could complement phagemids pMAB29 and pMAB77 in producing phagemid virion, and that these phage had significantly higher display than when using helper phage M13K07. The increase in display level with Phaberge was greater for pMAB29 (170 to 310-fold) than with pMAB77 (5 to 7-fold). The reason why substituting helper phage gives a greater improvement for pMAB29 than for pMAB77 is probably that pMAB77 gives better display than pMAB29 (Section B.1.1), and there is less room for further improvement with pMAB77.

For pMAB77, we obtained similar production of phagemid virions with Phaberge and M13K07, but for pMAB29, production was lower when using Phaberge. The reason for the lower production might be that in the case of Phaberge the assembly of infectious virions is critically dependent on phagemid-encoded g3p. Since pMAB29 appears to synthesize relatively little Fab-g3p (Table II A), production of phagemid virions would be constrained when using helper phage Phaberge (which is g3p-deficient) but not constrained with M13K07 (g3p-sufficient). pMAB77 likely has a higher synthesis of Fab-g3p than does pMAB29 (Table II A) and therefore, the number of virions would not be dependent upon helper phage-encoded g3p. Also, the same data suggest mutation that was introduced when creating Phaberge did not have a substantial effect on the assembly of infectious phagemid virion: The fact that production of phagemid virion was similar in the case of Phaberge+pMAB77 and M13K07+pMAB77 suggests that the mutation Q350amber does not have a severe polar effect.

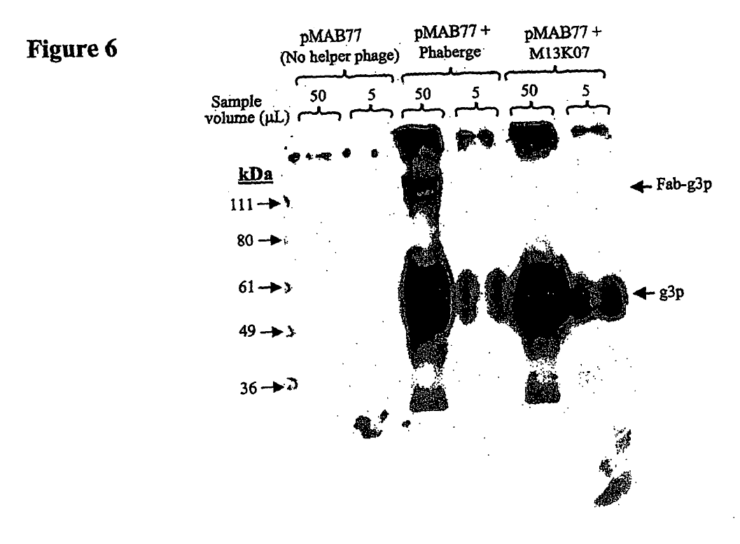

A Western blot experiment was also performed to test if Phaberge increases the display of Fab-g3p on phagemid virions (FIG. 6). In agreement with ELISA data (Table III, experiments 1 and 2A), we found that the combination of pMAB77+Phaberge yielded a more prevalent Fab-g3p band than did virions prepared by and pMAB77+M13K07.

It was also tested if Phaberge restrict its function to assembling only insert-containing phagemids into functional virions. Phagemid pMAB87 was used, which is identical to pMAB77, except that it lacks V_{H} and V□C□ inserts and has a translational stop codon immediately 5′ of gene 3. As shown in Table III, experiment 2B, the combination of Phaberge and pMAB87 gave too few infectious phagemid virion to be accurately determined, but the combination of M13K07 and pMAB87 gave at least 10⁴ times more virions. Although Phaberge and pMAB87 did not produce infectious virions, phage particles were still be detected by anti-phage sandwich ELISA. These may be either non-infectious phagemid virion or Phaberge virions, remaining from the time of infection.

This experiment also indicates that the present vector system does not have significant leakiness in g3p-production. If g3p production had occurred by any means (i.e. the stop codon of either the helper phage gene 3 or phagemid gene 3 had mutated to a sense-codon), it would have resulted in production of infectious phagemid virion, but this was apparently not to be the case.

1.5 Using Phaberge in Library Biopanning

In addition to testing functionality in model systems using a single phagemid vector, we also tested whether or not Phaberge can be used with a donor-derived phagemid library to isolate antigen-specific clones.

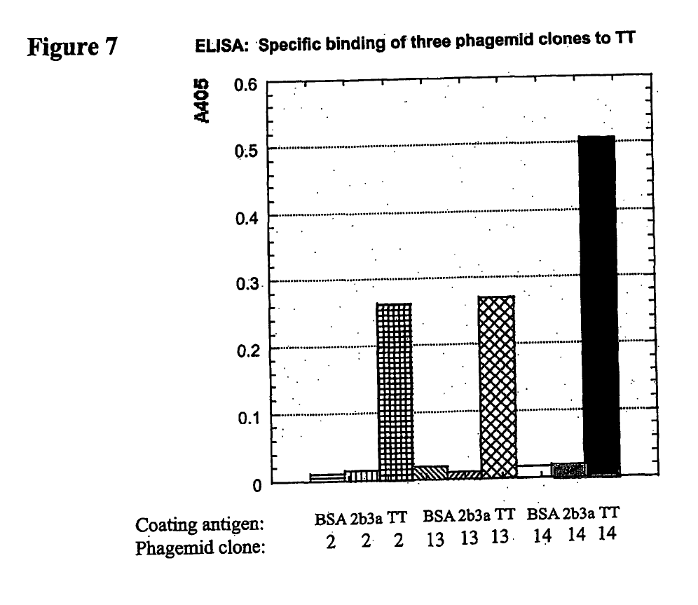

Section A.1.6 outlines the construction of a pMAB87-based library, and biopanning to obtain TT-specific clones. Four rounds of biopanning were performed and resulted in a 2,900-fold increase in the virion out-put: input ratio. This fact, as well as an ELISA of the selected virion-population (data not shown) suggested that a TT-specific phagemid population had been isolated. A sizeable proportion of selected clones were found to have full-length Fab inserts. Five randomly selected clones were subjected to DNA sequencing, which indicated three unique isolates (Table IV). These three unique clones showed significant binding to TT in whole-phage ELISA, but no significant binding to either of two control antigens: BSA and the human platelet protein GPIIbIIIa (FIG. 7).

In summary, it was found that Phaberge gave higher viral display level than did M13K07. Also, Phaberge discriminated between different types of phagemid: Only insert-containing, not insert-less phagemid was efficiently packaged into functional, infectious virions. Also, Phaberge has utility in isolating antigen-specific Fab clones from a library.

Although this invention disclosure describes display of immunoglobulin Fab fragments on phage, the same innovation can, with minimal modification, be applied to display of virtually any protein of interest.

Two other minimal modifications, which are apparent from this work, can also be used to further improve the functionality of this innovation: First, the ColE1/pUC region of phagemid pMAB87 can be replaced by a pBR322, as was done in vectors pMAB93 and pMAB103. Based on testing of functionality of pMAB103 (Section B1.1), we predict such vectors to be superior to pMAB87 in library biopanning work. Second, it is likely that counter-selection against insert-less clones can be made even more effective by modifying pMAB87 (or pMAB93): It is possible that in the current system (vectors Phaberge and pMAB87) only the presence of V_{H} is critical for generating infectious phagemid virions. If so, a modification could be made so that in addition to V_{H}, V_{L}C_{L} inserts would also be required, e.g: If a transcriptional terminator was engineered into the V_{L}C_{L} cloning site of pMAB87 (or pMAB93), then both this terminator, and the translational stop codon at the V_{H} cloning site would presumably have to be replaced by inserts to produce a virion carrying functional gene 3 protein.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Weiss, G., et al., Rapid mapping of protein epitopes by combinatorial alanine scanning. Proc. Natl. Acad. Sci. USA, 2000. 97(16): p. 8950.
2. Wilson, D. and B. Finlay, Phage display: applications, innovations and issues in phage and host biology. Can. J. Microbiol., 1998. 44: p. 313.
3. Rodi, D. and L. Makowski, Phage-display technology—finding a needle in a vast molecular haystack. Curr. Opin. Biotechnol., 1999. 10(87).
4. Smith, G., Filamentous fusion phage: novel expression vectors that display cloned antigens on the virion surface. Science, 1985. 228(4705): p. 1315.
5. Griffiths, A., et al., Human anti-self antibodies with high specificity from phage display libraries. EMBO J., 1993. 12(2): p. 725.
6. McCafferty, J., Phage display: Factors affecting panning efficiency, in Phage display of peptides and proteins, B. Kay, J. Winter, and J. McCafferty, Editors. 1996, Academic Press Inc.: San Diego, Calif., USA. p. 261.
7. Rondot, S., et al., A helper phage to improve single-chain antibody presentation in phage display. Nature Biotechnology, 2001. 19: p. 75.
8. Bass, S., R. Greene, and J. Wells, Hormone phage: an enrichment method for variant proteins with altered binding properties. Proteins: Structure, Function and Genetics, 1990. 8: p. 309.
9. Pratt, D., H. Tzagoloff, and W. Erdahl, Conditional lethal mutants of the small filamentous coliphage M13. I. Isolation, complementation, cell killing and cistron action. Virology, 1966. 30(3): p. 397.
10. Pratt, D., H. Tzagoloff, and J. Beaudoin, Conditional lethal mutants of the small filamentous coliphage M13. II. Two genes for coat proteins. Virology, 1969. 39(1): p. 42.
11. Beck, E. and B. Zink, Nucleotide sequence and genome organisation of filamentous bacteriophages f1 and fd. Gene, 1981. 16(1-3): p. 35.
12. Fowlkes, D., et al., Multipurpose vectors for peptide expression on the M13 viral surface. Biotechniques, 1992. 13(3): p. 422.
13. Parmley, S. and G. Smith, Antibody-selectable filamentous fd phage vectors: affinity purification of target genes. Gene, 1988. 73(2): p. 305.
14. Kristensen, P. and G. Winter, Proteolytic selection for protein folding using filamentous bacteriophages. Folding Design, 1998. 3(5): p. 321.
15. Knappik, A., et al., Fully synthetic human combinatorial antibody libraries (HuCAL) based on modular consensus frameworks and CDRs randomized with trinucleotides. J. Mol. Biol., 2000. 296: p. 57.
16. Barbas-III, C., et al., In vitro evolution of a neutralizing human antibody to human immunodeficiency virus type 1 to enhance affinity and broaden cross-reactivity. Proc. Natl. Acad. Sci, 1994. 91: p. 3809.
17. Hoogenboom, H., et al., Multi-subunit proteins on the surface of filamentous phage: methodologies for displaying antibody (Fab) heavy and light chain. Nucl. Acids Res., 1991. 19(15): p. 4133.
18. McGregor, D. and S. Robins, External surface display of proteins linked to DNA-binding domains. Analytical Biochemistry, 2001. 294(2): p. 108.
19. Ausubel, F., et al., Current protocols in molecular biology. 1997: John Wiley & Sons, Inc.
20. Sambrooke, J., E. Fritsch, and T. Maniatis, Molecular cloning. A laboratory manual. Second edition ed. 1989, Cold Spring Harbor: Cold Spring Harbor Laboratory Press.
21. Gigliotti, F., L. Smith, and R. A. Insel, Reproducible production of protective human monoclonal antibodies by fusion of peripheral blood lymphocytes with a mouse myeloma cell line. Journal of Infectious Diseases, 1984. 49(1).
22. Esposito, G., E. Scarselli, and C. Traboni, Phage display of a human antibody against Clostridium tetani toxin. Gene, 1994. 148(1): p. 167.
23. Vieira, J. and J. Messing, Production of single-stranded plasmid DNA. Methods in Enzymology, 1987. 153(D): p. 3.
24. Horton, R. M., et al., Engineering hybrid genes without the use of restriction enzymes: gene splicing by overlap exptension. Gene, 1989. 77: p. 61.
25. Russel, M., S. Kidd, and M. Kelley, An improved helper phage for generating single-stranded plasmid DNA. Gene, 1986. 45(3): p. 333.
26. Marks, J., et al., By-passing immunization. Human antibodies from V-gene libraries displayed on phage. J. Mol. Biol., 1991. 222: p. 581.
27. Heyman, B., et al., An enzyme-linked immunosorbent assay for measuring anti-sheep erythrocyte antibodies. J. Immunol. Methods, 1985. 68: p. 193.
28. Sage, D. R. Chillemi, A C; Fingeroth, J D, A versatile prokaryotic cloning vector with six dual restriction enzyme sites in the polylinker facilitates efficient subcloning into vectors with unique cloning sites, Plasmid, 1998, 40, 2: pg. 164.
29. Smith G. P, Filamentous fusion phage: novel expression vectors that display cloned antigens on the virion surface. Science, 1985, 228, 4705: pg. 1315.
30. Hufton, S E; Moerkerk, P T; Muelemans, E V; deBruine, A; Arends, A; Hoogenboom, H R, Phase display of cDNA repertoires: the pVI display system and its applications for the selection of immunogenic ligands, J. Immunol. Methods, 1999, 231,1-2: pg. 39.
31. Demartis S; Huber, A; Viti, F; Lozzi, L; Giovannoni, L; Neri, P; Winter, G; Neri, D., A strategy for the isolation of catalytic activities from repertoires of enzymes displayed on phage, J. Mol. Biol., 1999, 286, 2: pg. 617.
32. Yazynin, S. et al., A new phagemid vector for positive selection of recombinants based on a conditionally lethal barnase gene FEBS LETTERS 1999, 452:351
33. Larocca, D. et al., Receptor-targeted gene delivery using multivalent phagemid particles. Molec. Ther., 2001, 3(4): 476
34. Rakonjac, J. et al., Filamentous phage infection-mediated gene expression: construction and propagation of the gIII deletion mutant helper phage R408d3. Gene, 1997, 198 (1-2):99
35. Harlow, E. and D. Lane, Immunoblotting, in Antibodies. A laboratory manual. 1988, Cold Spring Harbor. p. 471.
36. Beekwilder, J. et al. A phagemid vector using the E. coli phage shock promoter facilitates phage display of toxic proteins. Gene, 1999, 228: 2337.
Gailus, V. et al. The role of the adsorption complex in the termination of filamentous phage assembly. Res. Microbiol. 1994, 145(9): 699

38. Zinder, N. D., Resistance to colicins E3 and K induced by infection of bacteriophage f1. Proc. Natl. Acad. Sci., 1973, 70(11): 3160.

TABLE I
Comparison of capabilities of the invention with prior art (i)

| | | | Second | |
|---|---|---|---|---|
| | | First generation | generation | |
| | Phage | phagemid | phagemid | Current |
| | system (ii) | system(ii) | system (ii) | invention |
| Publications | Examples: | Examples: | [5, 7, 33] | In press, |
| | [4, 12, 13] | [15-18, 32] | | by patent |
| | | | | applicant |
| I: Easy to make large | No: − (ii) | Yes: + (ii) | Yes: + | Yes: + |
| libraries (≧10⁸ | | | | |
| clones)? | | | | |
| II: Vector system | A: No: − | A: No: − [15-17] | A: No: − | A: Yes: + |
| designed to: | | Yes: + [18, 32] | | |
| A: Minimize presence | B: Yes: + | B: No: − (iii) | B: No: − | B: Yes: + |
| of insert-less clones | | | | |
| in initial library? | | | | |
| B: Make insert-less | | | | |
| clones unable to | | | | |
| propagate? | | | | |
| III: Is foreign gene | Yes: − | No: + | No: + | No: + |
| constitutively | | | | |
| expressed? | | | | |
| IV: High level of | Yes: + | No: − | Yes: + | Yes: + |
| protein display on | | | | |
| phage? | | | | |
| V: Complicated | No: ++ | Somewhat: + | Yes: − | Somewhat: + |
| system? | | | | |
| VI: Leakiness of g3 | Not | Not applicable | Yes: − [5, 33] | No: + |
| synthesis from helper | applicable | | No: + [7] | |
| phage preps? | | | | |
| VII: Low yield of | Not | No: + | Yes: − | No: + |
| helper phage? | applicable | | | |

Footnotes:
(i): Table only compares current invention with other phage display systems that have comparable design and applicability/usage.
(ii): Advantages and disadvantages of the different PDT systems are indicated with +and −, respectively.
(iii): One first generation phagemid system [14] prevents propagation of insert-less phage clones but has very limited utility (Section 3.3.3).

TABLE II
EFFECT OF MULTIPLE PARAMETERS ON PHAGE PRODUCTION
AND DISPLAY

| | | |
|---|---|---|
| | Phage | Fab display |
| Parameter varied | production, %⁽²⁾ | on phage, %⁽³⁾ |

A: Phagemid construct and IPTG induction

| | | |
|---|---|---|
| pMAB29, no IPTG⁽¹⁾ | 100 | 100 |
| pMAB29, 1 mM IPTG (n = 2)⁽⁴⁾ | 100 | 100 |
| pMAB77, no IPTG (n = 5) | 150 | 2300 |
| pMAB66, no IPTG (n = 8) | 180 | 1400 |
| pMAB66, 1 mM IPTG (n = 2) | 160 | 90 |
| pMAB103, no IPTG (n = 3) | 600 | 2500 |

B: Helper phage

| | | |
|---|---|---|
| R408⁽¹⁾ | 100 | 100 |
| M13-K07 (n = 4) | 72 | 350 |
| VCS-M13 (n = 4) | 130 | 110 |

C: Bacterial host strain

| | | |
|---|---|---|
| XL-1 Blue MRF′⁽¹⁾ | 100 | 100 |
| SURE (n = 5) | 65 | 610 |
| TOP10F′ (n = 6) | 75 | 2800 |
| TG-1 (n = 3) | 1 | 700 |

D: Temperature at growth

| | | |
|---|---|---|
| 37° C.⁽¹⁾ | 100 | 100 |
| 30-32° C. (n = 5) | 130 | 790 |

Footnotes:
⁽¹⁾Standard condition: pMAB29 phagemid, no IPTG induction, XL-1 Blue MRF′ host strain, R408 helper phage and growth at 37° C. The phage content and the display of anti-TT was designated as being 100% for this standard condition.
⁽²⁾Production measured after the PEG precipitation method. For each parameter that was altered, we determined the number of CFU/mL as a percentage of that produced during the standard condition. From different repeat experiments, we calculated the geometrical mean of all percentages, which is the number presented in the Table.
⁽³⁾Display of anti-TT Fab on phage as measured by anti-TT ELISA, and normalized for different phage concentration in different preparations. As in (2), the number is the geometric mean of percentage for repeat experiments.
⁽⁴⁾Number of experiments in which altered condition was compared to standard condition.

TABLE III
TESTING FUNCTIONALITY OF A NOVEL VECTOR SYSTEM

| | | |
|---|---|---|
| Phagemid | Production | Display of anti-TT |

| and helper | of phagemid virion⁽¹⁾ | | Fab as normalized by⁽²⁾: | |
|---|---|---|---|---|
| phage used for | | Phage | | Phage |
| production of | | sandwich | | sandwich |
| phagemid virion | CFU/mL | ELISA | CFU/mL | ELISA |
| Experiment 1 | | | | |
| pMAB29, M13K07 | 2 × 10¹¹ | 360,000 | 100% | 100% |
| pMAB29, Phaberge | 4 × 10⁹ | 13,000 | 25,000% | 17,000% |
| pMAB29, no helper | <1 × 10³ | Not done | Not | Not |
| phage | | | applicable | applicable |
| Experiment 2A | | | | |
| pMAB77, M13K07 | 0.6 × 10⁹ | 1,500 | 100% | 100% |
| pMAB77, Phaberge | 0.4 × 10⁹ | 1,500 | 770% | 480% |
| pMAB77, no helper | <2 × 10⁴ | <5 | Not | Not |
| phage | | | detected | detected |
| Experiment 2B | | | | |
| pMAB87, M13K07 | 1.0 × 10⁹ | 1,000 | Not | Not |
| | | | detected | detected |
| pMAB87, Phaberge | <4 × 10⁴ | 200 | Not | Not |
| | | | detected | detected |
| pMAB87, no helper | <4 × 10⁴ | <5 | Not | Not |
| phage | | | detected | detected |

Footnotes:
⁽¹⁾Phagemid virions were produced by growing TOP10F′ hosts in the absence of IPTG or kanamycin. Production was measured after the PEG precipitation method. The production was measured either after by the CFU assay, or by anti-phage sandwich ELISA. In the latter case, the column lists the reciprocal of the ELISA titer that gave 25% of maximum A₄₀₅.
⁽²⁾The Table lists the display of anti-TT Fab, normalized for number of phagemid virion: The sample prepared with M13K07 was considered to be the standard, giving 100% display.

TABLE IV
Sequence analysis of Fab clones isolated from blood donor library

| CLONE # | V_{H-}segment | D_{H} | J_{H} | Vₖ segment | Jₖ |
|---|---|---|---|---|---|
| 2 and 5 | V3-21*01 | D2-21*02/inv | J4*02 | V3-20*01 | J1*01 |
| 13 and 16 | V3-23*01 | D6-25*01 | J6*02 | V3-20*01 | J3*01 |
| 14 | V3-21*02 | D5-24*01/inv | J4*02 | V1D-39*01 | J2*01 |

Five TT-specific Fab phagemid clones were subjected to DNA sequencing, and their V (D,) and J-segments aligned using IGMT's web site: http://imgt.cnusc.fr:8104/textes/vquest/ to obtain the most closely related germline gene segment.

1. A helper phage for phage display comprising a conditional mutation in a filamentous phage viral coat protein gene wherein the conditional mutation causes minimal or no polar effects to downstream genes.

2. The helper phage according to claim 1 wherein the viral coat protein gene is gene 3.

3. The helper phage according to claim 1 wherein the viral coat protein gene can only be expressed in a host with permissive genotype.

4. The helper phage according to claim 1 wherein the conditional mutation is an amber mutation.

5. The helper phage according to claim 2 wherein the conditional mutation is in the latter third of gene 3.

6. The helper phage according to claim 2 wherein the conditional mutation is proximal to the 3′ end of gene 3.

7. The helper phage according to claim 1 wherein the mutation is introduced in helper phage strain M13KO7.

8. The helper phage according to claim 6 wherein the amber mutation is at residue Q350 of the leader-less gene product.

9. A phagemid vector comprising:
gene 3 from filamentous bacteriophage; and
a cloning site for inserting a nucleic acid molecule therein in-frame with gene 3 for creating a g3p fusion protein when expressed.

10. The phagemid according to claim 9 further including a sequence feature that prevents g3p synthesis in the absence of an inserted nucleic acid molecule.

11. The phagemid of claim 10 wherein the sequence feature is an in-frame stop codon prior to gene 3.

12. The phagemid of claim 9 wherein the phagemid is pMAB87.

13. A phage display system comprising:
a helper phage for phage display comprising a conditional mutation in a filamentous phage gene 3 wherein the conditional mutation causes minimal or no polar effects to downstream genes; and
a phagemid vector comprising:
gene 3 from filamentous bacteriophage;
a cloning site for inserting a nucleic acid molecule therein in-frame with gene 3 for creating a g3p fusion protein when expressed; and
a sequence feature that prevents g3p synthesis in the absence of an inserted nucleic acid molecule.

14. A method of creating a phagemid display system, comprising:
providing a helper phage for phage display comprising a conditional mutation in a filamentous phage gene 3 wherein the conditional mutation causes minimal or no polar effects to downstream genes.
providing a phagemid vector comprising:
gene 3 from filamentous bacteriophage; and
a cloning site for inserting a nucleic acid molecule therein in-frame with gene 3 for creating a g3p fusion protein when expressed; and
infecting a bacterial host with the phagemid and the helper phage.

15. The method according to claim 14 wherein the phagemid further includes a sequence feature that prevents g3p synthesis in the absence of an inserted nucleic acid molecule.

16. A method of screening for compounds binding to a target molecule using a phagemid display system, comprising:
providing a helper phage comprising a conditional mutation in a filamentous phage gene 3 wherein the conditional mutation causes minimal or no polar effects to downstream genes;
providing a phagemid vector comprising:
gene 3 from filamentous bacteriophage;
a cloning site for inserting a nucleic acid molecule therein in-frame with gene 3 for creating a g3p fusion protein when expressed; and
at least one nucleic acid molecule encoding a peptide inserted into the cloning site in frame with gene 3; and
infecting a bacterial host capable of suppressing the conditional mutation with the phagemid and the helper phage;
recovering the phagemid and the helper phage;
infecting a non-suppressing bacterial host with the phagemid and the helper phage;
growing the non-suppressing bacterial host under conditions wherein the phagemid is expressed, thereby producing a phage display library;
incubating the target molecule and the phage display library under conditions which promote peptide binding; and
detecting peptide binding.

17. A nucleic acid molecule encoding a peptide capable of binding to a target molecule identified according to the method of claim 16.

18. A peptide capable of binding to a target molecule identified according to the method of claim 16.


SEQUENCE LISTING (20 sequences)

SEQ ID NO: 1 - DNA, 29 bp, Artificial
  PCR primer
ctggctttaatgaggatccattcgtttgt

SEQ ID NO: 2 - DNA, 29 bp, Artificial
  PCR primer
attcaacactctaagggagggaaggtaaa

SEQ ID NO: 3 - DNA, 33 bp, Artificial
  PCR primer
ctcccttagagtgttgaatgtcgcccttttgtc

SEQ ID NO: 4 - DNA, 21 bp, Artificial
  PCR primer
tgcttctgtaaatcgtcgcta

SEQ ID NO: 5 - DNA, 2686 bp, Artificial
  Cloning vector pUC19
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 6 - DNA, 3162 bp, Artificial
  Cloning vector pUC119
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 7 - DNA, 4523 bp, Artificial
  pHEN1
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacgcagccgctggattgttattactcgcggcccagccggccatggcccaggtgcagctgcaggtcgacctcgagatcaaacgggcggccgcagaacaaaaactcatctcagaagaggatctgaatggggccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 8 - DNA, 1479 bp, Artificial
  pTIM1
cttgcatgcaaattctatttcaaggagacagtcataatgaaatacctattgcctacggcagccgctggattgttattactcgcggcccagccggccatggcccaggtgcagctgcaggtcaccgtctcgagtggtggaggcggttcaggcggaggtggctctggcggtggcggatcggatatcgagctcactgagatcaaacgggcggccgcagaacaaaaactcatctcagaagaggatctgaatggggccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaa

SEQ ID NO: 9 - DNA, 1479 bp, Artificial
  pMAB2
cttgcatgcaaattctatttcaaggagacagtcataatgaaatacctattgcctacggcagccgctggattgttattactcgcggcccagccggccatggcccaggtgcagctgcaggtcaccgtctcgagtggtggaggcggttcaggcggaggtggctctggcggtggcggatcggatatcgagctcactgagatcaaacgggcggccgcagaacaaaaactcatctcagaagaggatctaaatggggctgcagcgactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaa

SEQ ID NO: 10 - DNA, 1543 bp, Artificial
  pMAB3
cttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgcctcgagtggtggaggcggttcaggcggaggtggctctggcggtggcggatcggatatcgagctcactgagatcaaacgggcggccgcagaacaaaaactcatctcagaagaggatctaaatggggctgcagcgactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaa

SEQ ID NO: 11 - DNA, 2790 bp, Artificial
  pMAB29
cttgcatgcaaattctatttcaaggagacagtcataatgaaatacctattgcctacggcagccgctggattgttattactcgcggcccagccggccatggctgacatccagatgacccagtctccatcctccctgtctgcatctgtaggagacagagtcatcatcacttgccgggcaagtcagagtattagcacctatttaaattggtatcagcagaaaccagggaaagcccctaaactcctgatctattatgcaaccaatttgcaaagtggggtcccatcaaggttcagtggcagtggatctgggacagatttcactctcaccatcagcagtctgcaacctgaagattttgcgacttattattgtcaacagagttccaacaccgtcactttcggccctgggaccaaagtggatatgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaattctagagtaaggaggcagtcataatgaagtaccttttgccaacggctgccgctggcttgttattgctcgcggcacagccggcaattgcccaggtgcagctggtgcagtctgggggaggcttggtacagcctggggggtccgtgagactctcctgtgcagcctctggattcagttttagcagctatgccatgagctgggtccgccaggctccagggatggggctggagtgggtcgcggctattagtgctagaggaactaccacatattatgcagactccgtgacgggccgattgaccatctccagagacaattccatgaacacgctatatctgcacttgaacagcctgagagccgaggacacggccgtttattactgtgcgaaagcgggaaaacagtggctggcccactactactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgcagaacaaaaactcatctcagaagaggatctaaatggggctgcagcgactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaa

SEQ ID NO: 12 - DNA, 939 bp, Artificial
  pMAB65
cttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgcctcgagtggtggaggcggttcaggcggaggtggctctggcggtggcggatcggatatcgagctcactgagatcaaacgggcggccgctagccctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 13 - DNA, 2186 bp, Artificial
  pMAB66
cttgcatgcaaattctatttcaaggagacagtcataatgaaatacctattgcctacggcagccgctggattgttattactcgcggcccagccggccatggctgacatccagatgacccagtctccatcctccctgtctgcatctgtaggagacagagtcatcatcacttgccgggcaagtcagagtattagcacctatttaaattggtatcagcagaaaccagggaaagcccctaaactcctgatctattatgcaaccaatttgcaaagtggggtcccatcaaggttcagtggcagtggatctgggacagatttcactctcaccatcagcagtctgcaacctgaagattttgcgacttattattgtcaacagagttccaacaccgtcactttcggccctgggaccaaagtggatatgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaattctagagtaaggaggcagtcataatgaagtaccttttgccaacggctgccgctggcttgttattgctcgcggcacagccggcaattgcccaggtgcagctggtgcagtctgggggaggcttggtacagcctggggggtccgtgagactctcctgtgcagcctctggattcagttttagcagctatgccatgagctgggtccgccaggctccagggatggggctggagtgggtcgcggctattagtgctagaggaactaccacatattatgcagactccgtgacgggccgattgaccatctccagagacaattccatgaacacgctatatctgcacttgaacagcctgagagccgaggacacggccgtttattactgtgcgaaagcgggaaaacagtggctggcccactactactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgctagccctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 14 - DNA, 1563 bp, Artificial
  pMAB64
cttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgcctcgagtggtggaggcggttcaggcggaggtggctctggcggtggcggatcggatatcgagctcactgagatcaaacgggcggccgctagcactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 15 - DNA, 2810 bp, Artificial
  pMAB77
cttgcatgcaaattctatttcaaggagacagtcataatgaaatacctattgcctacggcagccgctggattgttattactcgcggcccagccggccatggctgacatccagatgacccagtctccatcctccctgtctgcatctgtaggagacagagtcatcatcacttgccgggcaagtcagagtattagcacctatttaaattggtatcagcagaaaccagggaaagcccctaaactcctgatctattatgcaaccaatttgcaaagtggggtcccatcaaggttcagtggcagtggatctgggacagatttcactctcaccatcagcagtctgcaacctgaagattttgcgacttattattgtcaacagagttccaacaccgtcactttcggccctgggaccaaagtggatatgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaattctagagtaaggaggcagtcataatgaagtaccttttgccaacggctgccgctggcttgttattgctcgcggcacagccggcaattgcccaggtgcagctggtgcagtctgggggaggcttggtacagcctggggggtccgtgagactctcctgtgcagcctctggattcagttttagcagctatgccatgagctgggtccgccaggctccagggatggggctggagtgggtcgcggctattagtgctagaggaactaccacatattatgcagactccgtgacgggccgattgaccatctccagagacaattccatgaacacgctatatctgcacttgaacagcctgagagccgaggacacggccgtttattactgtgcgaaagcgggaaaacagtggctggcccactactactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgctagcactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 16 - DNA, 1182 bp, Artificial
  pMAB86
cttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgggcgcgcctagtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgctagccctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 17 - DNA, 1806 bp, Artificial
  pMAB87
cttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgggcgcgcctagtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgctagcactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaa

SEQ ID NO: 18 - DNA, 4912 bp, Artificial
  pMAB93
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacggcagccgctggattgttattactcgcggcccagccggccatggctctagagtaaggaggcagtcataatgaagtaccttttgccaacggctgccgctggcttgttattgctcgcggcacagccggcaattgggcgcgcctagtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgctagcactgttgaaagttgtttagcaaaacctcatacagaaaattcatttactaacgtctggaaagacgacaaaactttagatcgttacgctaactatgagggctgtctgtggaatgctacgggcgttgtggtttgcactggtgacgaaactcagtgttacggtacatgggttcctattgggcttgctatccctgaaaatgagggtggtggctctgagggtggcggttctgagggtggcggttctgagggtggcggtactaaacctccagagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagctagccatcaccaccatcatcactaataatgaaagcccgcctaatgagcgggcttttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 19 - DNA, 1440 bp, Artificial
  pMAB103
ggcccagccggccatggctgacatccagatgacccagtctccatcctccctgtctgcatctgtaggagacagagtcatcatcacttgccgggcaagtcagagtattagcacctatttaaattggtatcagcagaaaccagggaaagcccctaaactcctgatctattatgcaaccaatttgcaaagtggggtcccatcaaggttcagtggcagtggatctgggacagatttcactctcaccatcagcagtctgcaacctgaagattttgcgacttattattgtcaacagagttccaacaccgtcactttcggccctgggaccaaagtggatatgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaattctagagtaaggaggcagtcataatgaagtaccttttgccaacggctgccgctggcttgttattgctcgcggcacagccggcaattgcccaggtgcagctggtgcagtctgggggaggcttggtacagcctggggggtccgtgagactctcctgtgcagcctctggattcagttttagcagctatgccatgagctgggtccgccaggctccagggatggggctggagtgggtcgcggctattagtgctagaggaactaccacatattatgcagactccgtgacgggccgattgaccatctccagagacaattccatgaacacgctatatctgcacttgaacagcctgagagccgaggacacggccgtttattactgtgcgaaagcgggaaaacagtggctggcccactactactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaagcggccgc

SEQ ID NO: 20 - DNA, 8669 bp, Artificial
  clone 4B
gtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctcccttagagtgttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggacggatcgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgcccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccccctgaaggtgtgggcctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaac